(19) United States
(12) Patent Application Publication
(10) Pub. No.: US 2021/0298606 A1
(43) Pub. Date: Sep. 30, 2021

(54) Acousto-Optical Sensors for MRI Safety Evaluation

(71) Applicant: Georgia Tech Research Corporation, Atlanta, GA (US)

(72) Inventors: Ozgur Kocaturk, Rockville, IL (US); Fahrettin Levent Degertekin, Atlanta, GA (US); Yusuf S. Yaras, Atlanta, GA (US)

(21) Appl. No.: 17/259,033

(22) PCT Filed: Jul. 9, 2019
(86) PCT No.: PCT/US2019/040982

§ 371 Date: Jan. 8, 2021

Related U.S. Application Data
(60) Provisional application No. 62/695,260, filed on Jul. 9, 2018.

(51) Int. Cl. A61B 5/00; G01R 29/08; A61B 5/055; G01K 11/3206; G01R 33/28; G01R 33/36

(52) U.S. Cl. CPC A61B 5/0097; G01R 29/0885; G01R 33/3692; G01K 11/3206; G01R 33/288; A61B 5/055

(57) ABSTRACT

Methods are disclosed herein for measuring local E-fields, B-fields, and/or temperature effects of an MRI scan utilizing an acousto-optical sensor. A method includes positioning the acousto-optical sensor at a location of a body or phantom; receiving, with an antenna of the acousto-optical sensor, MRI RF energy localized at the first location; interrogating, with a light source, and via an optical fiber, an acousto-optical sensor region of the acousto-optical sensor; detecting, with a photodetector, interrogation light reflected from the acousto-optical sensor region; and outputting one or more signals corresponding to the detected interrogation light reflected from the acousto-optical sensor region. The one or more signals can correspond to an E-field, a B-field, and/or a temperature of the received MRI RF energy at the first location. Additional methods can include mapping results of multiple measurements around an implant.

### CROSS-REFERENCE TO RELATED APPLICATIONS

This application claims priority to U.S. Provisional Patent Application No. 62/695,260, entitled “Acousto-optical Sensors for MRI Safety Evaluation,” filed 9 Jul. 2018, the contents of which are also incorporated by reference in their entirety as if set forth in full. This application is also related to U.S. patent application Ser. No. 15/303,002, entitled “Interventional MRI Compatible Medical Device, System, and Method,” filed 10 Apr. 2015, and published as U.S. Patent Publication No. US2017/0143234 on 25 May 2017, the contents of which are also incorporated by reference in their entirety as if set forth in full.

### GOVERNMENT SUPPORT

This invention was made with government support under Grant No. EB017365 awarded by the National Institutes of Health. The government has certain rights in the invention.

### FIELD

The disclosed technology relates generally to MRI safety evaluation, and more specifically to acousto-optical sensors for measuring localized radio frequency (RF) electric and magnetic fields and associated temperatures indicative of specific absorption rates (SAR).

### BACKGROUND

Magnetic resonance imaging (MRI) utilizes a combination radio frequency (RF) waves and magnetic fields to non-invasively create detailed images of tissues, organs, etc., of a subject's body. During an MRI examination, three types of fields are typically employed to produce three dimensional images: (1) a strong static magnetic field (typically up to 3 T) that generates a net proton magnetization vector in the human body; (2) a gradient magnetic field, typically in the frequency range of 100 to 1,000 Hz, which is utilized to localize aligned protons inside the body, thus allowing spatial reconstruction of tissue sections into images; and (3) a radio frequency (RF) electromagnetic wave, typically in the frequency range of 10 to 400 MHz, which is utilized to energize the magnetization vector allowing conversion of tissue properties into images. Different levels of contrast are based on the different magnetic properties and physical structure of the biological tissues.

While it is well established that the non-ionizing electromagnetic energy produced during an MRI scan can impose fewer health risks compared with the ionizing radiation associated with x-rays, local RF-induced currents and/or specific absorption rates (SAR) of tissue, fluid, implants, etc., under MRI can lead to hot spots and burns, as discussed in “Biological Effects and Safety in Magnetic Resonance Imaging: A Review,” (Hartwig, et al., Int J Environ Res Public Health, 2009), which is incorporated herein by reference in its entirety.

Certain types of monitoring equipment and/or implantable devices can include elongated conductors that can heat up in the presence of the MRI fields (for example, due to RF-induced eddy currents) and cause burns upon contact with the patient. U.S. Patent Publication No. US2017/0143234, which is incorporated herein by reference in its entirety, discloses a device for active device visualization under MRI and provides certain solutions to address the risk of RF-induced heating by using acousto-optical sensors and optical fiber.

Another major recognized mechanical risk associated with MRI is the presence of metallic (ferromagnetic) devices that can be subject to the attractive and rotational forces caused by the static field. Thus, the presence of certain devices and implants, such as catheters, heart valve prostheses, coronary artery stents, aortic stent grafts, pacemakers, implantable defibrillators, etc., can create thermal and mechanical safety hazards in the MRI environment.

To assess the potential dangerous biological effects associated with MRI on biological tissues, there is a need for small MRI dosimeters that can measure localized field strengths, associated thermal responses, and/or evaluate effects caused by the presence of an implant during MRI scans. However, currently available dosimeters are bulky, and typically include cabling with conductors that can heat up and/or have associated output signals corrupted by the RF field induced over the conductive lines. These dosimeters, are, therefore, not suitable for in-situ measurements of RF fields over the patient's body. Certain exemplary implementations are disclosed herein to address the above-referenced needs and risks.

In addition to SAR measurements during MRI scans, there is also a need for mapping RF magnetic fields, so-called B1 fields, both during MRI scans as well as during the testing of implants for MRI safety classification and qualification. For example, the test methods for ISO (the International Organization for Standardization) standards such as ISO/TS 10974:2018 “Assessment of the safety of magnetic resonance imaging for patients with an active implantable medical device,” requires that B1 fields be measured. Accurate and local measurements of RF Electric (E) field and B1 field measurements are critical for MRI safety determination.

Miniature RF E field and B1 field sensors placed on flexible catheters or surgical tubes are also desired in characterizing and mapping the RF induced heating effects during the manufacture and testing of medical implants such as cardiac pacemakers and neurostimulation devices implanted in the central nervous system (brain and spinal cord). The RF induced currents on these implant wires depend on their exact shape and location in the body (or phantoms during testing). Mapping the variation of RF induced currents over these wires with a high spatial resolution during testing these devices under MRI fields through E field and B1 field measurements help determine the safety of particular implant and preferred shape. Currently available bulky dosimeters are not suitable for this purpose.

During MRI scans or MRI device safety testing, simultaneous measurement of RF field components at multiple points on the patient body or along the implant wiring is desired as these field components can show significant spatial variation.

### BRIEF SUMMARY

Briefly described, certain exemplary implementations of the disclosed technology include acousto-optical sensors for measuring localized RF fields and associated temperatures indicative of specific absorption rates and/or presence of implantable devices. Some or all of the above needs may be addressed by certain implementations of the disclosed technology.

According to an exemplary implementation of the disclosed technology, a method is provided for measuring a local E-field during an MRI scan. The method includes: providing an acousto-optical sensor, the acousto-optical sensor including: an optical fiber including a distal end; an acousto-optical sensor region disposed towards the distal end of the optical fiber, the acousto-optical sensor region including an electro-mechanical conversion assembly comprising: an antenna configured to receive E-field radio-frequency (RF) energy and to produce a corresponding electrical signal; and an ultrasonic transducer in mechanical communication with the acousto-optical sensor region, wherein the ultrasonic transducer is in electrical communication with the antenna, and wherein the ultrasonic transducer is configured to elastically modulate the acousto-optical sensor region by acoustic waves generated responsive to an electrical signal corresponding to RF energy received at the antenna. The method includes positioning the acousto-optical sensor at a first location of a body; receiving, with the antenna, MRI RF energy localized at the first location; interrogating, with a light source, and via the optical fiber, the acousto-optical sensor region; detecting, with a photodetector, interrogation light reflected from the acousto-optical sensor region; and outputting an E-field signal corresponding to the detected interrogation light reflected from the acousto-optical sensor region, wherein the E-field signal corresponds to an E-field of the received MRI RF energy at the first location.

According to another exemplary implementation of the disclosed technology, a method is provided for measuring a local B-field during an MRI scan. The method includes providing an acousto-optical sensor, the acousto-optical sensor including: an optical fiber including a distal end; an acousto-optical sensor region disposed towards the distal end of the optical fiber, the acousto-optical sensor region including an electro-mechanical conversion assembly comprising: a loop antenna configured to receive B-field radio-frequency (RF) energy and to produce a corresponding electrical signal; and an ultrasonic transducer in mechanical communication with the acousto-optical sensor region, wherein the ultrasonic transducer is in electrical communication with the loop antenna, and wherein the ultrasonic transducer is configured to elastically modulate the acousto-optical sensor region by acoustic waves generated responsive to an electrical signal corresponding to RF energy received at the loop antenna. The method includes positioning the acousto-optical sensor at a first location of a body; receiving, with the loop antenna, MRI RF energy localized at the first location; interrogating, with a light source, and via the optical fiber, the acousto-optical sensor region; detecting, with a photodetector, interrogation light reflected from the acousto-optical sensor region; and outputting a B-field signal corresponding to the detected interrogation light reflected from the acousto-optical sensor region, wherein the B-field signal corresponds to a B-field of the received localized MRI RF energy at the first location.

According to another exemplary implementation of the disclosed technology, a method is provided for attaching one or more acousto-optical sensors on a surface of a body. The method includes: providing an acousto-optical sensor, the acousto-optical sensor including a first optical fiber including a distal end; a first acousto-optical sensor region disposed at a first position towards the distal end of the first optical fiber, the first acousto-optical sensor region including an electro-mechanical conversion assembly comprising: a first antenna configured to receive radio-frequency (RF) energy and to produce a corresponding first electrical signal; and a first ultrasonic transducer in mechanical communication with the first acousto-optical sensor region, wherein the first ultrasonic transducer is in electrical communication with the first antenna, and wherein the first ultrasonic transducer is configured to elastically modulate the first acousto-optical sensor region by acoustic waves generated responsive to an electrical signal corresponding to RF energy received at the first antenna. The method includes positioning the acousto-optical sensor on a surface of a body, and securing the acousto-optical sensor to the surface of the body

According to another exemplary implementation of the disclosed technology, a method is provided for measuring a local temperature and one or more of a local E-field or a local B-field during an MRI scan. The method includes providing a combined thermo-optical and acousto-optical sensor, the combined thermo-optical and acousto-optical sensor including: an optical fiber including a distal end; a fiber Bragg grating (FBG) disposed towards the distal end of the optical fiber; an electro-mechanical conversion assembly comprising: an antenna configured to receive radio-frequency (RF) energy and to produce a corresponding electrical signal; and an ultrasonic transducer in mechanical communication with the FBG, wherein the ultrasonic transducer is in electrical communication with the antenna, and wherein the ultrasonic transducer is configured to elastically modulate the FBG by acoustic waves generated responsive to an electrical signal corresponding to RF energy received at the antenna. The method includes positioning the combined thermo-optical and acousto-optical sensor at a first location of a body; receiving, with the antenna, MRI RF energy at the first location; interrogating, with a light source, and via the optical fiber, the FBG; detecting, with a photodetector, an interrogation signal based on light reflected from the FBG; processing the interrogation signal; and outputting, based on processing the interrogation signal, one or more of: a field signal corresponding to the received MRI RF energy at the first location; and a temperature signal corresponding to a wavelength shift in the light reflected from the FBG.

According to another exemplary implementation of the disclosed technology, a method is provided for measuring local temperature and one or more of a local E-field or local B-field during an MRI scan. The method includes providing a combined thermo-optical and acousto-optical sensor, the combined thermo-optical and acousto-optical sensor including: an optical fiber including a distal end; a fiber Bragg grating (FBG) disposed towards the distal end of the optical fiber; a GaAs-based temperature detector disposed at the distal end of the optical fiber; and an electro-mechanical conversion assembly comprising: an antenna configured to receive radio-frequency (RF) energy and to produce a corresponding electrical signal; and an ultrasonic transducer in mechanical communication with the FBG, wherein the ultrasonic transducer is in electrical communication with the antenna, and wherein the ultrasonic transducer is configured to elastically modulate the FBG by acoustic waves generated responsive to an electrical signal corresponding to RF energy received at the antenna. The method includes positioning the combined thermo-optical and acousto-optical sensor at a first location of a body; receiving, with the antenna, MRI RF energy at the first location; interrogating, with a light source, and via the optical fiber, the FBG and the temperature detector; detecting, with a photodetector, an interrogation signal based on light reflected from the FBG and the temperature detector; processing the interrogation signal; and outputting, based on processing the interrogation signal, one or more of: a field signal corresponding to the received MRI RF energy at the first location; and a temperature signal corresponding to a spectrum shift in the light reflected from the temperature detector.

According to another exemplary implementation of the disclosed technology, a method is provided for mapping effects of MRI over an implant. The method includes: providing an acousto-optical sensor, the acousto-optical sensor including: an optical fiber including a distal end; an acousto-optical sensor region disposed towards the distal end of the optical fiber, the acousto-optical sensor region including an electro-mechanical conversion assembly comprising: an antenna configured to receive radio-frequency (RF) energy and to produce a corresponding electrical signal; and an ultrasonic transducer in mechanical communication with the acousto-optical sensor region, wherein the ultrasonic transducer is in electrical communication with the antenna, and wherein the ultrasonic transducer is configured to elastically modulate the acousto-optical sensor region by acoustic waves generated responsive to an electrical signal corresponding to RF energy received at the antenna. The method includes mounting at least a distal end of the acousto-optical sensor in a housing configured to move the acousto-optical sensor around at least a portion of the implant; sequentially positioning the mounted acousto-optical sensor at a plurality of locations within a body in a region of the implant; sequentially receiving, with the antenna, MRI RF energy localized at the corresponding plurality of locations; sequentially interrogating, with a light source, and via the optical fiber, the acousto-optical sensor region; sequentially detecting, with a photodetector, corresponding interrogation light reflected from the acousto-optical sensor region; and sequentially outputting field signals corresponding to the sequentially detected interrogation light reflected from the acousto-optical sensor region, wherein the field signals correspond to the received MRI RF energy at the plurality of locations.

According to another exemplary implementation of the disclosed technology, a method is provided for simultaneously mapping effects of MRI over an implant. The method includes: providing an acousto-optical sensor, the acousto-optical sensor including: an optical fiber including a distal end; a plurality of acousto-optical sensor regions disposed towards the distal end of the optical fiber, each of the plurality of acousto-optical sensor regions including an electro-mechanical conversion assembly comprising: a fiber Bragg grating (FBG); an antenna configured to receive radio-frequency (RF) energy and to produce a corresponding electrical signal; and an ultrasonic transducer in mechanical communication with the FBG wherein the ultrasonic transducer is in electrical communication with the antenna, and wherein the ultrasonic transducer is configured to elastically modulate the FBG by acoustic waves generated responsive to an electrical signal corresponding to RF energy received at the antenna. The method further includes mounting at least a distal end of the acousto-optical sensor in a housing configured to position and move the acousto-optical sensor around at least a portion of the implant; positioning the mounted acousto-optical sensor within a body in a region of the implant such that each of the plurality of acousto-optical sensor regions are disposed at a plurality of corresponding locations; receiving, with each antenna of the plurality of acousto-optical sensor regions, MRI RF energy localized at the plurality of corresponding locations; interrogating, with a light source, and via the optical fiber, the acousto-optical sensor; detecting, with a photodetector, corresponding interrogation light reflected from the acousto-optical sensor; and outputting field signals corresponding to the detected interrogation light reflected from the acousto-optical sensor, wherein the field signals correspond to the received MRI RF energy at the plurality of corresponding locations.

Other implementations, features, and aspects of the disclosed technology are described in detail herein and are considered a part of the claimed disclosed technology. Other implementations, features, and aspects can be understood with reference to the following detailed description, accompanying drawings, and claims.

### BRIEF DESCRIPTION OF THE FIGURES

Reference will now be made to the accompanying figures and flow diagrams, which are not necessarily drawn to scale, and wherein:

FIG. 1A is an illustration of an acousto-optical based MRI dosimeter device, according to an exemplary embodiment of the disclosed technology.

FIG. 1B is an illustration of a typical use-case for the acousto-optical based MRI dosimeter device, according to an exemplary embodiment of the disclosed technology.

FIG. 2A is a schematic illustration of an acousto-optical sensor system, according to an exemplary implementation of the disclosed technology.

FIG. 2B illustrates the reflectivity of a fiber Bragg grating (FBG) as a function of wavelength. The reflected light intensity may be modulated by the FBG via a corresponding Bragg wavelength modulation of the FBG as induced by the received RF signal, as shown in FIG. 4A.

FIG. 3 depicts an electric field antenna, in accordance with an exemplary implementation of the disclosed technology.

FIG. 4 is an exemplary plot of a Bragg wavelength of an FBG as a function of temperature.

FIG. 5 illustrates part of an exemplary acousto-optical based MRI dosimeter device in which a separate FBG within the same optical fiber may be utilized for sensing temperature, in accordance with an exemplary implementation of the disclosed technology.

FIG. 6A depicts an experimental setup for characterizing induced E-field and/or current distributions around and along an insulated lead wire under test.

FIG. 6B is a graph showing the experimentally measured induced current output of the acousto-optical sensor.

FIG. 7A depicts a portion of an enhanced acousto-optical marker device having an acoustic resonator formed by a notch in the optical fiber near the FBG region, according to an exemplary implementation of the disclosed technology.

FIG. 7B depicts a portion of another embodiment of an enhanced acousto-optical marker device having an acoustic resonator formed by deposition of material on the optical fiber, according to an exemplary implementation of the disclosed technology.

FIG. 8 depicts an acoustic resonator utilizing radial mode resonances of the optical fiber, in accordance with certain exemplary implementations of the disclosed technology.

FIG. 9 depicts the resonance frequency shift as a function of (a) electrode thickness, and (b) piezoelectric layer thickness for the radial mode acoustic resonator as shown in FIG. 8, in accordance with certain exemplary implementations of the disclosed technology.

FIG. 10 is a flow diagram of a method of measuring a local E-field during an MRI scan, according to an exemplary embodiment of the disclosed technology.

FIG. 11 is a flow diagram of a method of measuring a local B-field during an MRI scan, according to an exemplary embodiment of the disclosed technology.

FIG. 12 is a flow diagram of a method of attaching one or more acousto-optical sensors on a surface of a body, according to an exemplary embodiment of the disclosed technology.

FIG. 13 is a flow diagram of a method of measuring local temperature and one or more of a local E-field or local B-field during an MRI scan, according to an exemplary embodiment of the disclosed technology.

FIG. 14 is a flow diagram of a method of measuring local temperature and one or more of a local E-field or local B-field during an MRI scan, according to an exemplary embodiment of the disclosed technology.

FIG. 15 is a flow diagram of a method of mapping effects of MRI over an implant, according to an exemplary embodiment of the disclosed technology.

FIG. 16 is a flow diagram of a method of simultaneously mapping effects of MRI over an implant, according to an exemplary embodiment of the disclosed technology.

### DETAILED DESCRIPTION

The disclosed technology relates to sensor devices, systems, and methods that may be utilized for evaluating safety in certain magnetic resonance imaging (MRI) applications. Certain exemplary implementations of the disclosed technology may include acousto-optical sensors for measuring localized RF fields and/or associated temperature increases indicative of specific absorption rates (SAR). Certain aspects of the disclosed technology are related to U.S. Patent Publication No. US2017/0143234, the contents of which is incorporated by reference in its entirety as if set forth in full.

U.S. Patent Publication No. US2017/0143234 describes an acousto-optical (AO) catheter probe incorporating an active receiver that can modulate interrogation light at a frequency of the localized MRI gradient field. The reflected and modulated light can be utilized to determine the location of the probe. The probe can include a receiver coil in communication with a piezoelectric transducer that is coupled to an acousto-optical sensor region of an optical fiber. For example, the acousto-optical sensor region can include a fiber Bragg grating (FBG). The optical fiber serves as a transmission line that enables the elimination of typically elongated lead wire conductors that can heat up (and damage surrounding tissue) in the presence of the MRI equipment's electromagnetic RF field. The piezoelectric transducer is directly in contact with the optical fiber over the FBG region and generates acousto-optical modulation signals directly on the fiber. Using a thin film piezoelectric layer directly deposited on the fiber partially or fully over the circumference, the elastic waves generated by the piezoelectric layer may be cylindrically focused on the core of the optical fiber where it is most effective. This technique presents efficient acousto-optical modulation at a target frequency for locating the receiver coil position.

The disclosed technology includes certain advancements and improvements, that when combined with the technology disclosed in U.S. Patent Publication No. US2017/0143234 may be utilized to produce an improved device that can address certain challenges, limitations, and issues associated with prior devices.

RF-induced heating can impact patients during an MRI as the locally induced current distribution over the patient's body can cause temperature rise leading to hot spots and burns. This is because the specific absorption rate (SAR) values reported by the MRI manufacturers are obtained over phantoms, and the estimated SAR values may not reflect the real situation with the variety of the patients scanned.

Certain features and devices disclosed herein can include an MRI dosimeter configured to measure local radio frequency (RF)-induced currents and/or associated increases in temperature (essentially SAR) over an area on the patient's body with a low profile, non-invasive and MRI compatible sensor. Certain exemplary implementations of the disclosed technology can include methods and techniques that enable measurements of local electric and/or magnetic fields and/or the associated temperature effects thereof as MRI fields interact with bodily implants, associated lead conductors, phantoms, etc. Certain exemplary implementations of the disclosed technology may be utilized for implantable device evaluation (for example, within phantoms) with increased resolution and/or reduced RF interference.

Certain elements of the disclosed technology may further utilize electrical-to-mechanical energy conversion via a piezoelectric transducer for receiver signal extraction. In accordance with certain exemplary implementations, acousto-optical modulation on the optical fiber (i.e., mechanical-to-optical signal conversion) may be utilized for signal detection and transmission. In certain exemplary implementations, a fiber Bragg grating (FBG) may be utilized in conjunction with the piezoelectric transducer. The resulting combination may yield improved devices that can include MRI safe active receivers and/or location markers without conducting transmission lines and without compromising mechanical performance.

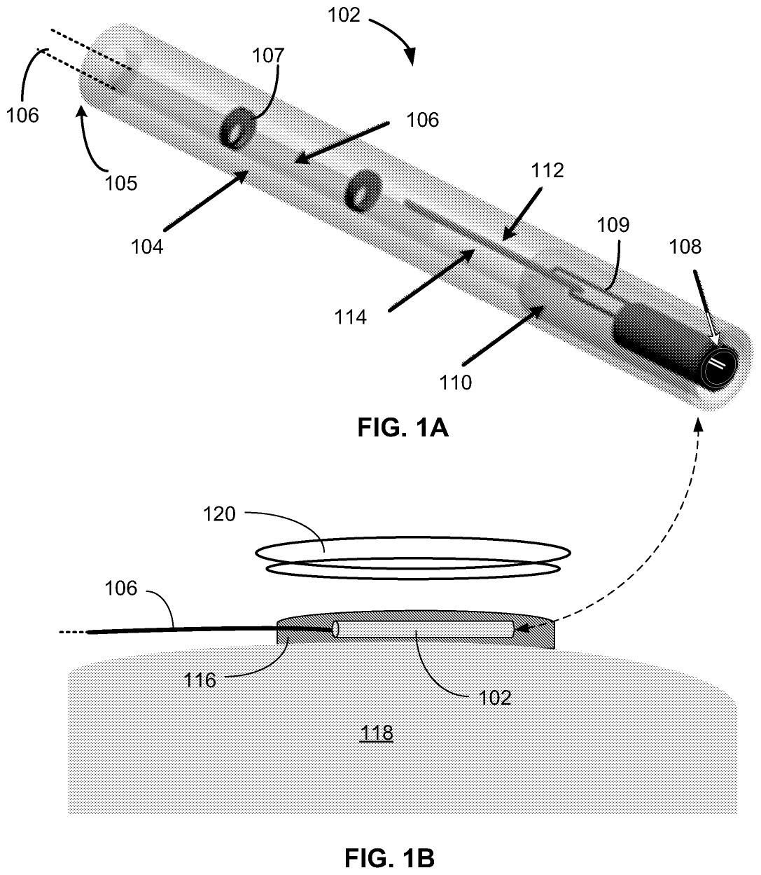

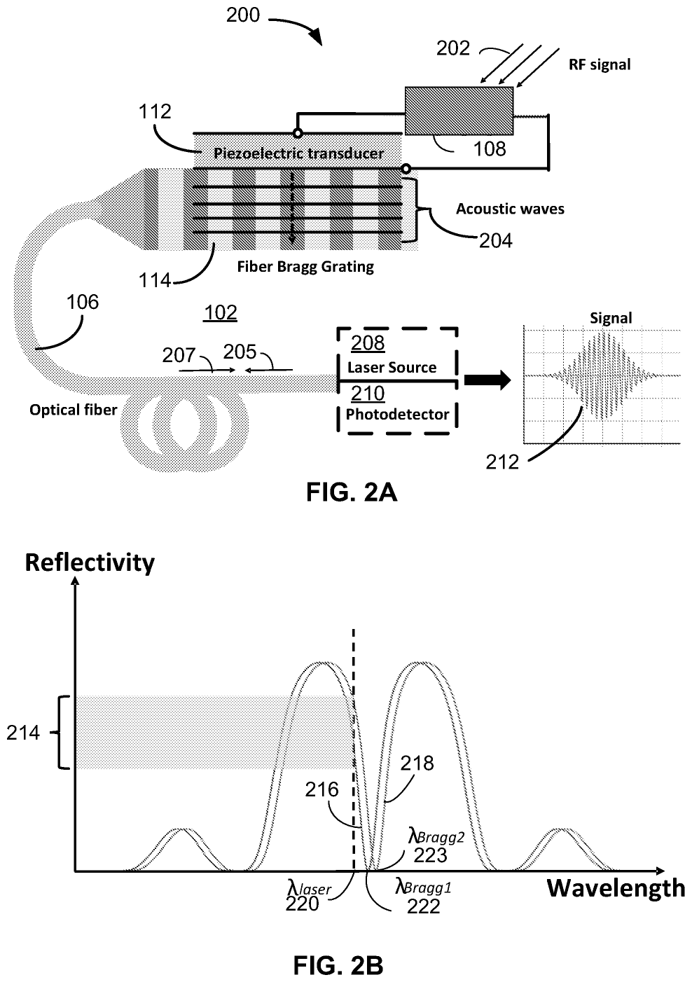

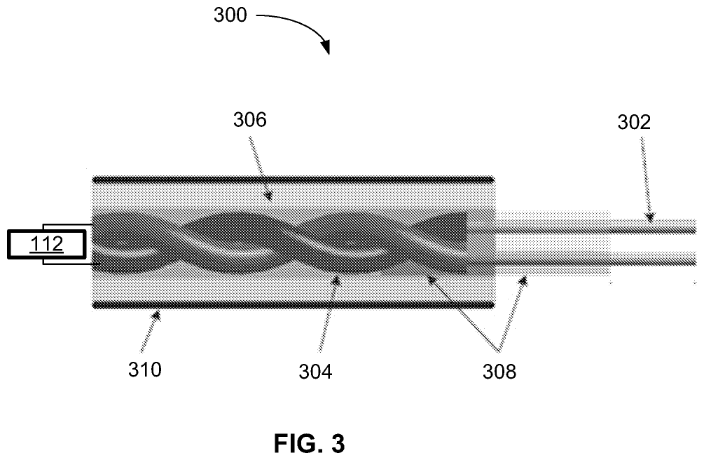

FIG. 1A is an illustration of an acousto-optical MRI dosimeter device 102 for real-time MRI measurements, according to an exemplary embodiment of the disclosed technology. Some or all of the components associated with the device 102 may be housed in a micro-lumen 104. In some implementations, the micro-lumen 104 may be surgical tubing or the like. In certain exemplary implementations, the device 102 may utilize an optical fiber 106 with a fiber Bragg grating (FBG) 114 disposed near the end of the optical fiber 106. The optical fiber 106 may receive interrogation light (for example, from an external swept laser) which may interact with the FBG 114 to provide a reflected optical signal that is sent back through the optical fiber 106 to an external detector. As will be further explained below with reference to FIG. 2A and FIG. 2B, a receiver antenna 108 may be utilized to sense the local electric (E field) and/or magnetic field (B1 field). In certain exemplary implementations, the receiver antenna may be configured to measure electric field (E-field) strength and may be embodied as a two-electrode probe configuration 300 discussed below with respect to FIG. 3. In certain exemplary implementations, the receiver antenna 108 may be in the form of a coil for measuring magnetic field strength, or RF magnetic field called B1 field as discussed in U.S. Patent Publication No. US2017/0143234, which is incorporated herein by reference as if presented in full.

The receiver antenna 108 may be connected to an ultrasonic transducer 112, which may be in intimate contact with the FBG 114 region. In certain exemplary implementations, the ultrasonic transducer 112 may be a piezoelectric transducer that is driven by the receiver antenna 108. Certain exemplary implementations of the device 102 can include a rigid connection support 110 for holding the receiver antenna 108 and/or the associated short interconnections 109 to the transducer 112. In certain implementations, the optical fiber 106 may be held in place with one or more O-rings 107 or spacers.

FIG. 1B is an illustration of a typical use-case for the acousto-optical based MRI dosimeter device 102 for measuring local RF fields (and/or temperature response) responsive to signals produced by MRI coils 120. According to an exemplary embodiment of the disclosed technology, the device 102 can be embedded in an adhesive bandage 116 and may be attached to a patient's body 118. In other implementations, the dosimeter device 102 may be inserted within a phantom (not shown), for example, to evaluate the MRI field interaction with a device under test, such as an implantable device. In this embodiment, and as discussed above, the optical fiber 106 may extend out the proximal end 105 to an external light source (such as a swept laser) and a detector.

Certain implementations may include additional antenna connected in parallel with the piezoelectric transducer 112. In certain exemplary implementations, one or more antennae may be utilized to provide additional sensors along the length of the device 102.

FIG. 2A is a schematic illustration of an acousto-optical sensor system 200, according to an exemplary implementation of the disclosed technology. The system 200 may include the MRI dosimeter device 102 (as discussed above) in communication with a laser source 208 and photodetector assembly 210. The antennae 108 may receive RF signals 202 (from the MRI system) to produce acoustic waves 204 via the piezoelectric transducer 112. In this exemplary embodiment, a Fiber Bragg Grating (FBG) 114 may be used to enhance the detection sensitivity of the generated acoustic waves 204. The acoustic waves 204 may modulate the Bragg wavelength of the FBG 114. (Note, the illustrated FBG 114 is expanded in FIG. 2A for clarity).

In certain exemplary implementations, light 205 emitted from the laser source 206 (such as a swept laser) may traverse the optical fiber 106 and a portion of the incident light may be reflected (as a function of wavelength) by the FBG 114 such that reflected light 207 is modulated corresponding to the RF signal 202 frequency, the wavelength of the laser source 208, and as a function of the reflectivity curve (see FIG. 2B) of the FBG 114 to produce an output signal 212. In certain exemplary implementations, the frequency of the output signal 212 may correspond to the position(s) of the antenna 108 in the MRI gradient field.

According to another exemplary implementation of the disclosed technology (not shown), the piezoelectric transducer 112 may be mechanically coupled directly to the optical fiber 106. In this embodiment, the corresponding acoustic waves 204 produced in the optical fiber 106 and generated by the piezoelectric transducer 112 may modulate the elastic properties of the fiber at the RF frequencies (via the AO effect) corresponding to the RF signal 202, which in turn can be detected by laser-based interferometric sensing. Because the optical fiber 106 is not conductive, the sensor is immune to RF interference and heating along its length.

FIG. 2B illustrates reflectivity curves 216, 219 of the FBG 114 as a function of wavelength for two different acoustic wave 204 intensities, with continued reference to the exemplary components shown in FIG. 2A. In certain exemplary implementations, the reflected light 207 intensity may be modulated by the FBG 114 via the received RF signal 202 and the piezoelectric transducer 112. The light 205 output of the laser source 208 (for this illustration) may have a wavelength 220 fixed at or near a maximum slope of the reflectivity curve 216 to maximize the signal output. As the acoustic wave 204 modulates the FBG 114, the Bragg wavelength (and associated curves 216, 219) of the FBG 114 may oscillate between a first Bragg wavelength 222 and a second Bragg wavelength 223, thus modulating the intensity of the reflected light 207 over a range 214 corresponding to the RF field strength and resulting in the wavelength shift in the reflectivity curves 216, 219.

FIG. 3 depicts an electric field (E-field) antenna 300, in accordance with an exemplary implementation of the disclosed technology. The use of such an antenna for E-field measurements may be useful since the specific absorption rate (SAR) may be proportional to the squared E-field. In accordance with certain exemplary implementations of the disclosed technology, the antenna 300 may be connected to the piezoelectric transducer 112, as discussed with reference to FIG. 2A, and the E-field sensed with the antenna 300 may be utilized to modulate the piezoelectric transducer 112 (and associated FBG). In an exemplary embodiment, the antenna 300 may include two straight distal tip electrodes 302 approximately 3 mm in length, approximately 0.2 mm in diameter, and separated by approximately 0.8 mm. However, other lengths, diameters, spacing, and shapes (including dipole, monopole, and microstrip configurations) may be utilized without departing from the scope of the disclosed technology.

In certain exemplary implementations, the distal tip electrodes 302 may be connected directly to the piezoelectric transducer 112. In other exemplary implementations (as shown in FIG. 3), a short section of twisted pair insulated conductors 304 may connect the distal tip electrodes 302 with the piezoelectric transducer 112. In certain exemplary implementations, a tube or similar structure 306 may house and/or secure the distal tip electrodes 302 and/or associated conductors 304. In certain exemplary implementations, a non-conductive insulator material 308 such as silicone may be further utilized to secure and/or protect a portion of the distal tip electrodes 302 and/or associated conductors 304. In accordance with certain exemplary implementations of the disclosed technology, a shielding 310 such as foil may be utilized to cover a portion of the structure 306.

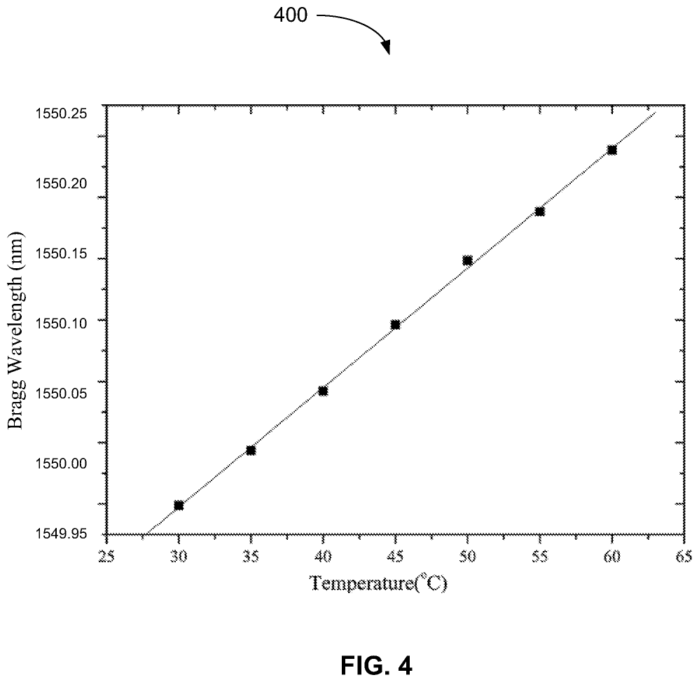

FIG. 4 is a graph 400 depicting the Bragg wavelength (nm) of an FBG as a function of temperature (° C.) for a particular FBG. Accordingly, a temperature change acting on the FBG can result in a shift in reflectivity curve (like the shift depicted in FIG. 2B), which may be detected. The temperature dependence results from changes of the refractive index of the fiber as well as from thermal expansion of the glass material. Since the temperature change acts on the FBG at a much slower rate (<100 Hz) compared with the RF-induced wavelength shift (>1 MHz), the temperature change may be determined by measuring the slowly varying wavelength shift.

In accordance with certain exemplary implementations of the disclosed technology, in addition to sensing a local E-field, the acousto-optical MRI dosimeter device 102 can also have an integrated temperature sensor for RF safety monitoring. In certain exemplary implementations, this temperature sensor can be the same FBG 114 as discussed above. When the same FBG 114 is used for both RF field (E-field and/or B-field) and temperature sensing, the slowly varying wavelength shift due to temperature change can be used as an indicator of temperature. Alternatively, the low slope region of the FBG reflectance spectrum (such as shown in FIG. 2B) can be used for temperature measurement while the highly sensitive n-phase shifted region of the FBG spectrum may be used for the fast RF field measurements.

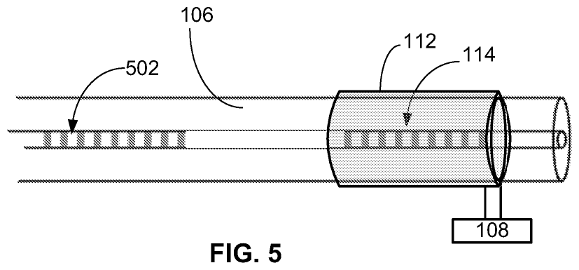

FIG. 5 illustrates part of an acousto-optical based MRI dosimeter device (such as the device 102 as discussed above) in which a separate FBG 502 within the same optical fiber may be utilized for sensing temperature, in accordance with an exemplary implementation of the disclosed technology. In certain exemplary implementations, the temperature sensing FBG 502 may have a Bragg wavelength that differs from the Bragg wavelength of the RF sensing FBG 114. In certain exemplary implementations, both the temperature sensing FBG 502 and the RF sensing FBG 114 may be written on the same optical fiber. In certain implementations, the overall reflectance spectrum of the temperature sensing FBG 502 may be interrogated using the same interrogation laser as is used for interrogating the RF sensing FBG 114. In certain exemplary implementations, a different laser coupled to the same optical fiber 106 may be utilized to separately interrogate the temperature sensing FBG 502. In other implementations, a temperature-induced reflectance spectrum shift over a given wavelength range or spectrum slope can be similarly interrogated.

FIG. 5 also depicts a certain configuration for the piezoelectric transducer 112. In this implementation, a thin conductive inner electrode may be deposited in a cylindrical ring around the optical fiber 106. Piezoelectric material, such as ZnO may be deposited on the inner electrode. A conductive outer electrode may be deposited on the piezoelectric layer. In certain exemplary implementations, the inner and outer electrode may be connected to the antenna 108.

In yet another exemplary embodiment, the temperature sensing FBG can be disposed on a separate optical fiber and packaged next to the RF sensing FBG. In this case, the temperature sensing FBG can be interrogated using a separate system, but the readings from both the temperature sensing FBG and the RF sensing FBG can be synchronized and displayed together to observe the correlation between the sensors.

Yet in another alternative embodiment, a temperature sensor may be integrated into the acousto-optical based MRI dosimeter device, for example, by attaching the temperature sensor at the end of the optical fiber. In certain exemplary implementations, a reflection spectrum corresponding the localized temperature may be monitored to provide absolute temperature information, for example, as discussed in “A New Fiber Optical Thermometer and its Application for Process Control in Strong Electric, Magnetic, and Electromagnetic Fields,” Roland et al, Sensor Letters, Vol. 1, pp. 93-98, 2003, which is incorporated by reference herein as if presented in full. Such gallium arsenide (GaAs) crystal-based optical temperature sensors are well known, but they have not previously been integrated with acousto-optical RF sensors.

In accordance with certain exemplary implementations of the disclosed technology, a broadband light source or a swept laser source may be coupled into the fiber and the reflectance spectrum may be measured. Since GaAs behaves as a temperature sensitive cut-off filter in which the crystal absorbs some light and transmits other light depending on temperature, the characteristic edge, or transition wavelength, between the reflected and transmitted spectrum is directly related to the band gap energy, and hence the absolute temperature. In certain exemplary implementations, photo energy from the light source may excite electrons from the valence to the conduction band of the semiconductor crystal. The required amount of energy is equal to the so-called band-gap energy, Egap. The well-known underlying principle of operation is based on the temperature dependence of the band gap of the direct (zone center) GaAs intrinsic gap; Egap=1.423 eV, corresponding to 872 nm at 300° K; dEgap/dT=−0.452 meV/° K at 300° K. According to an exemplary implementation of the disclosed technology, a GaAs sensor crystal may be placed in a medium of unknown temperature and the reflectance spectrum may be measured. In certain exemplary implementations, the wavelength position of the characteristic edge may be analyzed to determine the temperature.

RF-induced heating over interventional devices during an MRI scan is subject to guidelines and regulations by the US Food and Drug Administration by the International Electrotechnical Commission. The guidelines are summarized in Table 1.

TABLE 1
IEC and FDA guidelines on SAR and heating in human MRI studies.

| | Whole-Body | | Heat | | Head, Trunk | | Extremities | |
|---|---|---|---|---|---|---|---|---|
| Limit | Average | | Average | | Local SAR | | Local | |
| IEC (6-minute average) | | | | | | | | |
| Normal (all patients) | 2 W/kg | (0.5° C.) | 3.2 | W/kg | 10 | W/kg | 20 | W/kg |
| First level (supervised) | 4 W/kg | (1° C.) | 3.2 | W/kg | 10 | W/kg | 20 | W/kg |
| Second level (IRB approval) | 4 W/kg | (>1° C.) | >3.2 | W/kg | >10 | W/kg | >20 | W/kg |

| | | | |
|---|---|---|---|
| Localized heating limit | 39° C. in 10 g | 38° C. in 10 g | 40° C. in 10 g |

| | | | | |
|---|---|---|---|---|
| FDA | 4 W/kg for | 3 W/kg for | 8 W/kg in 1 g | 12 W/kg in 1 g |
| | 15 min | 10 min | for 10 min | for 5 min |

Most of the frequently used active implantable medical devices (AIMD) such as cardiac pacemakers, defibrillators and neurostimulators should be evaluated in terms of their availability for MRI studies. Electromagnetic field and implantable device interactions may induce current/heating. Implant lead malfunctions, unexpected pacing behavior, inappropriate shocks, ventricular defibrillation, and even deaths have been reported. The American Society of Testing Materials (ASTM) designates devices as MR safe, MR conditional, and MR unsafe through ASTM F2053-13 “Standard Practice for Marking Medical Devices and Other Items for Safety in the Magnetic Resonance Environment” Standard. “MR Safe” medical devices are composed of nonmetallic materials and systems. Therefore, it is very difficult to design an implantable pacemaker or ICD system that can be assigned as “MR Safe”. “MR Conditional” refers to devices which pose no known hazards to patients when MRI scans are performed with specific conditions and the approval of a device requires strict definition of these conditions. Thus, each medical device should be tested extensively to determine safe MRI scan conditions.

The presence of ferromagnetic device components in the strong static and gradient magnetic fields of the MR system can lead to unintentional movement or vibration of the implant devices. Accordingly, implantable medical devices should be manufactured and tested using MR Compatible materials. The long conductors of pacing leads may behave as antennas and interact with RF waves during MR scans. Through this interaction, the transfer of RF energy to pace leads may induce heat and the amount of temperature rise is dependent on factors including pulse sequence parameters (i.e. flip angle, TR/TE ratio), the whole body averaged, and local specific absorption rates (SAR) associated with a given sequence, lead factors such as lead design, length, placement configuration, and orientation.

Many factors such as lead length, lead configuration within the phantom, phantom shape and MR surface coil position affect induced heating over pace leads. Mechanical and electrical requirements of lead design have prompted multilayer and non-homogenous device design—such as alternating winding configurations of lead wires. It has been shown that increasing the lead insulation thickness along the lead wire can cause increased heating in that section. Therefore, it is very important to have a heating profile of overall lead wires instead of just the lead tip. Another study has shown that when the lead wire is near the phantom edge, the RF induced heating over the lead wire also increases.

The interaction of an AIMD and the RF field of an MR scanner is a complicated process and depends on AIMD design, AIMD location within the body, bird cage, and surface coils design, patient size, anatomy and tissue properties. Depending on the specific conditions, in vivo temperature rise variation may span several orders of magnitude. For example, a pacemaker lead implanted in an anatomical region that receives minimal RF exposure may have lower RF induced heat compare to a shorter neurostimulator lead implanted in a region receiving higher RF exposure. Therefore, FDA asks all AIMD manufacturers to test their devices according to an additional standard, the ISO/TS 10974 “Assessment of the safety of magnetic resonance imaging for patients with an active implantable medical device.” The tests for this standard include both E-field and B1-field measurement, which can be achieved using dipole, monopole, and/or microstrip antennas for E-field, and coil or loop-type antennas which can be wire wound or printed on printed circuit boards or different dielectric substrates for B-field. However, current commercially available probes tend to have a large diameter probe tip, a rigid probe shaft, and bulky circuitry, which can limit tests, such as measuring the RF induced current variation along a small profile lead wires. Also, because the long probe shaft may interact with RF waves, users should calibrate their probes for each MRI in vitro phantom tests. Certain exemplary implementations of the disclosed technology can provide a compact, flexible and RF immune sensor to map the E-field or current distribution over these AIMD leads, including the distal tip electrode and the overall lead shaft.

The RF induced heating issue can be particularly problematic when the patients have implanted medical devices having metallic components, which can locally amplify or change the induced E-fields. The current and temperature distribution over these implants should be carefully measured for each device in a variety of configurations to mimic clinical use conditions within phantoms. For example, a lead may be tested by moving an RF sensor along the length of the lead, or by passing the lead through a sensing coil and moving the sensing coil along the lead.

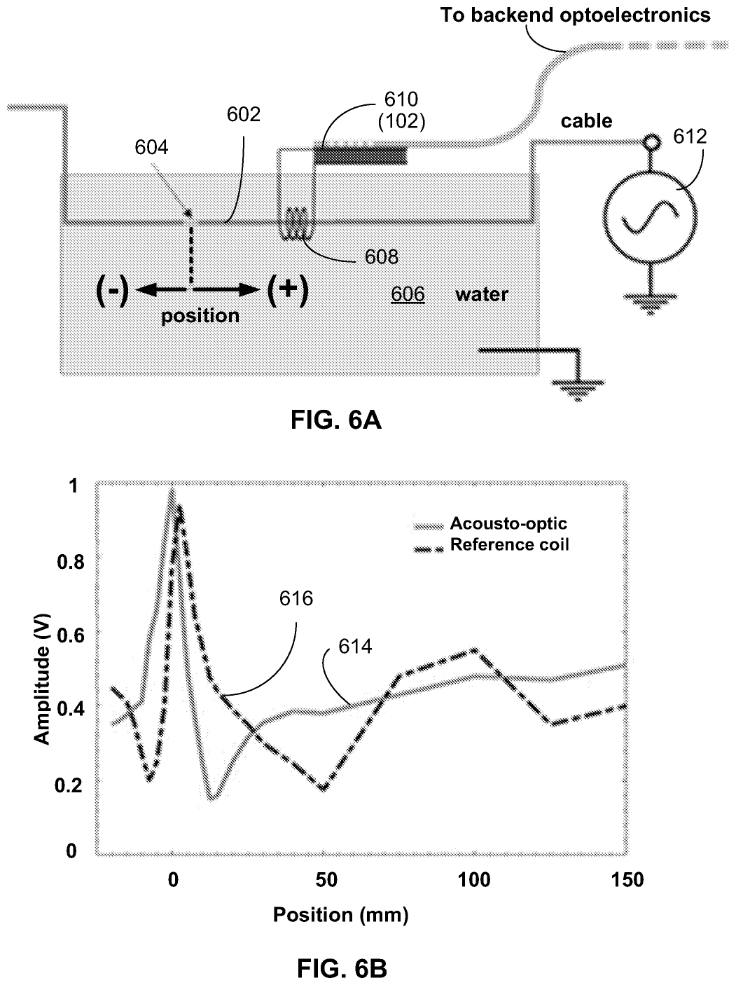

FIG. 6A depicts an experimental setup for characterizing induced E-field and/or current distributions around and along an insulated lead wire 602 under test. The insulator has a small sub-mm opening 604, exposing the inner conductor of the lead wire 602. In this experimental setup, the lead wire 602 is immersed in a water tank phantom 606 and the lead wire 602 is passed through a coil 608 of an acousto-optical sensor 610. In this experimental setup, the lead wire 602 is then fed a 64 MHz RF signal 612 to simulate induced E-field and current distributions over the wire 602 during MRI scan. The acousto-optical sensor 610 is advanced along the length of the lead wire 602 including the exposed opening 604 at the 0 mm position.

FIG. 6B is a graph showing the experimentally measured induced current output 614 of the acousto-optical sensor 610 (which may be the same or similar to the acousto-optical based MRI dosimeter device 102 discussed herein). The graph also shows the measured induced current output 616 obtained by connecting the coil 608 with a coaxial cable. Both signals 614, 616 show a sharp peak at the location of the uninsulated gap section 604 where there is current crowding and a significant change in the induced current. In addition, there is a spatial variation of the current. These experimental results show that the acousto-optical sensor 610 can map the distribution of the induced current along a conductor and/or on a human body, where the current induced in the coil 608 is related to the B1 field and hence the acousto-optical sensor 610 provides a measurement of this important RF field component. Thus, the acousto-optical sensors disclosed herein may be utilized as MRI dosimeters for obtaining local SAR measurements.

Certain exemplary implementations of the disclosed technology may be utilized to evaluate active implantable medical device leads for MRI safety evaluation. Furthermore, as indicated in FIG. 6B, RF-induced E field measurement at a single point on a conductor may not provide accurate information on induced currents on a long wire. Knowledge of the spatial variation of the induced currents along conductive implant components may be critical for high field MRI scanners such as 3 T, 7 T, and 11.4 T.

In clinical use, RF-induced heating of tissue surrounding an active implantable medical device can be caused by increased local SAR that arises from induced current along the conductive structures. Furthermore, eddy currents induced by the MRI gradient field can cause heating of the implantable device and/or associated lead wires. According to an exemplary implementation of the disclosed technology, acousto-optical based MRI dosimeter devices (such as the device 102 discussed herein) may be used to map/measure the SAR locally over the skin.

In accordance with certain exemplary implementations of the disclosed technology, the acousto-optical based MRI dosimeter disclosed herein may include an integrated temperature sensor to provide temperature information. In certain exemplary implementations, the disclosed technology may allow investigation of factors, such as the relationship between the SAR and the squared electric field. Other factors may be investigated utilizing the disclosed technology, such as B field coupling, standing wave formation, and gradient coil effects.

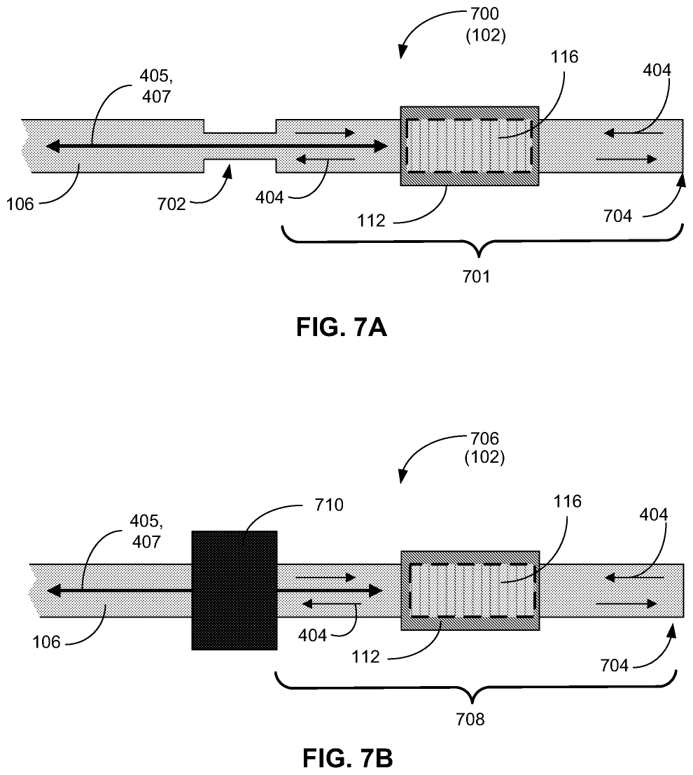

FIG. 7A depicts a portion of an enhanced acousto-optical based MRI dosimeter device 700, according to an exemplary embodiment (with the receiver antenna omitted for clarity). The enhanced device 700 may include an acoustic resonator 701 formed by a notch 702 in the optical fiber 106 near the (proximal side) region of the FBG 114. In accordance with certain exemplary implementations of the disclosed technology, the notch 702 feature may be incorporated into and utilized in the acousto-optical based MRI dosimeter devices 102 (as described above) to at least partially trap the acoustic wave(s) 404 generated by the piezoelectric transducer 112 and to enhance the amplitude of the acoustic wave(s) 404 that interact with the FBG 114. As discussed above with respect to the acousto-optical based MRI dosimeter devices 102, the FBG 114 may be covered by a piezoelectric material to form the piezoelectric transducer 112, which may be connected to an RF antenna (not shown). In certain exemplary implementations, the notch 702 feature may be formed by removing at least a portion of cladding from the optical fiber 106 to create an acoustic discontinuity which may reflect the acoustic wave(s) 404 back towards the distal end 704 of the optical fiber 106, where the acoustic wave(s) 404 may also be reflected to interact with the FBG 114.

FIG. 7B depicts a portion of another embodiment of an enhanced acousto-optical based MRI dosimeter device 706 having an acoustic resonator 708 formed by application of material on the optical fiber 106, near the (proximal side) region of the FBG 114 to form a coating or ring feature 710 on the optical fiber 106. In accordance with certain exemplary implementations of the disclosed technology, the ring feature 710 may be incorporated into and utilized in the acousto-optical based MRI dosimeter device 102 (as described above) to at least partially trap the acoustic wave(s) 404 generated by the piezoelectric transducer 112 and to enhance the amplitude of the acoustic wave(s) 404 that interact with the FBG 114. As discussed above with respect to the acousto-optical based MRI dosimeter device 102, the FBG 114 may be covered by a piezoelectric material to form the piezoelectric transducer 112, which may be connected to an RF antenna (not shown). In certain exemplary implementations, the ring feature 710 may create an effective acoustical discontinuity which may reflect the acoustic wave(s) 404 back towards the distal end 704 of the optical fiber 106, where the acoustic wave(s) 404 may also be reflected to further interact with the FBG 114.

In accordance with certain exemplary implementations of the disclosed technology, the one or more of the features of the enhanced devices 700, 706, as discussed above with reference to FIG. 7A and/or FIG. 7B may be utilized to increase the sensitivity of the device 102 by creating an acoustic resonator structure to make more efficient use of the acoustic energy generated by the receiver antennae and piezoelectric transducer 112. The notch 702 and/or the ring feature 710 discontinuities may enhance acoustic wave 404 reflections, acting as a mirror for the acoustic waves 404 and effectively trapping the acoustic energy between the distal end 704 of the optical fiber 116 and the notch 702 or ring feature 710. Such acoustic wave 404 reflections may increase the amplitude of the strains generated in the FBG 114 region as acoustic energy builds up and result in larger acousto-optical modulation for given available electrical power from the receiver antennae, therefore increasing the sensitivity.

In certain exemplary implementations, the distance between the distal end 704 and the notch 702 or ring feature 710 can be optimized based on the acoustic fields generated at the Larmor frequency. Since these geometrical features are far away from the core region of the optical fiber 106, the propagating light 405, 407 and its interaction with the FBG 114 may not be adversely affected by the acoustic resonators 701, 708. There are many different ways of implementing such acoustic resonators 701, 708 such as reflectors formed by small but periodic perturbations of the structure of the optical fiber 106, where the periodicity is determined by the wavelength of the acoustic waves 404. In certain exemplary implementations, a quality factor of acoustic resonators 701, 708 may be adjusted so that the bandwidth of the device is still large enough to cover the typical MRI signal bandwidth of about 100 kHz.

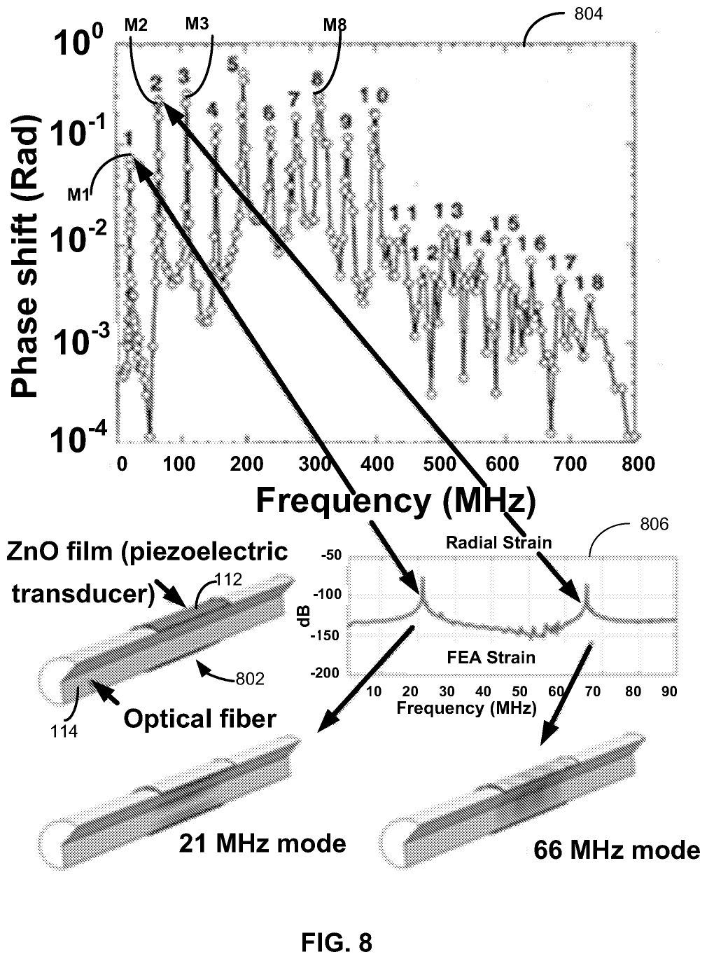

FIG. 8 depicts cutaway views of an acoustic resonator 802 that may utilize radial mode resonances of an optical fiber 116, in accordance with certain exemplary implementations of the disclosed technology. Certain implementations of the enhanced acousto-optical based MRI dosimeter device may utilize certain radial acoustic resonance modes of the optical fiber 106 in the region of the FBG 114 under the piezoelectric transducer 112, for example, by forming the piezoelectric transducer 112 using a thin film piezoelectric material sandwiched between thin film metal layers. The publication: “High-performance optical phase modulation using piezoelectric ZnO-coated standard telecommunication fiber,” Gusarov et al., Journal of Lightwave Technology, vol. 14, pp. 2771-2777, December 1996, which is incorporated herein by reference as presented in full, discusses acousto-optical phase shift measurements 804 on a standard, Zinc oxide (ZnO) coated 125 μm diameter standard communication grade optical fiber. This structure has multiple radial mode resonance frequencies which are already very close to the Larmor frequencies: M1:21.6 MHz (˜0.55 T), M2:66.3 MHz (˜1.5 T), M3:110 MHz (close to 3 T), M8: 316 MHz (˜7 T). By changing the thickness of the piezoelectric coating and the metal layers forming the piezoelectric transducer 112 over the FBG 114, the radial resonances can be tuned to the desired Larmor frequency for enhanced sensitivity without a need for other modifications of the optical fiber 106 (such as the modifications discussed above with respect to FIG. 7A or FIG. 7B). The thickness values for the piezoelectric layers and metal layers can be calculated according to well-known coupled vibration formulations for a composite structure (such as the optical fiber 106 and the thin film piezoelectric transducer 112) as discussed in the abovementioned Gusarov reference. In this implementation, the section of the optical fiber 106 in the region of FBG 114 under the piezoelectric transducer 112 becomes an acoustic resonator without modifying other sections of the optical fiber. Given that the bandwidth required for MRI RF signal is about 100 kHz for 1.5 T, these resonances can provide sufficient bandwidth.

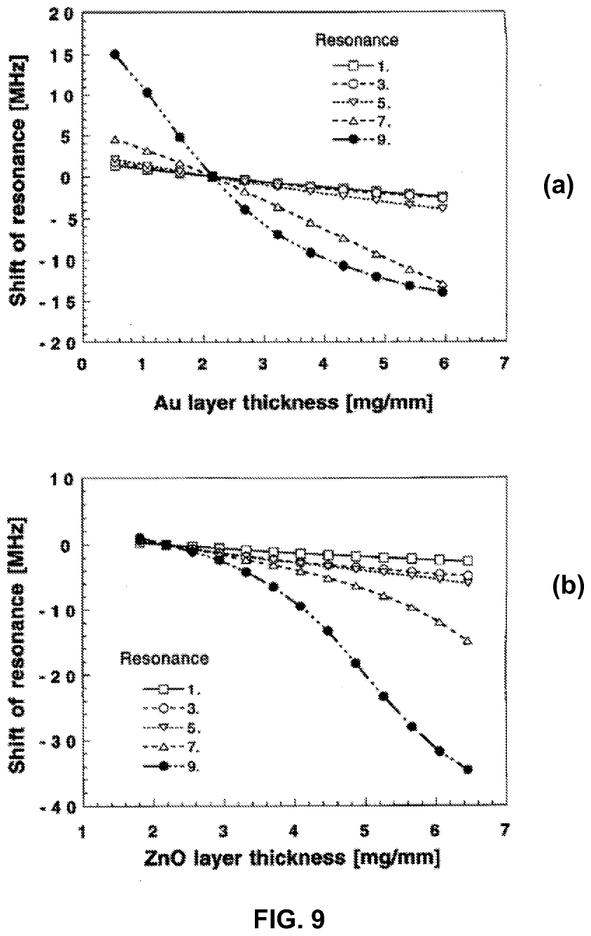

FIG. 9 depicts the resonance frequency shift (MHz) as a function of (a) electrode thickness (top graph), and (b) piezoelectric layer thickness (bottom graph) for the radial mode acoustic resonator as shown in FIG. 8, in accordance with certain exemplary implementations of the disclosed technology, and as discussed in the abovementioned Gusarov reference. In certain exemplary implementations of the disclosed technology, the radial mode acoustic resonator (such as resonator 802 shown in FIG. 8) may be configured with a length ranging from 0.5 mm to 10 mm. In certain exemplary implementations, the length may range from 10 mm to 20 mm. In certain exemplary implementations of the disclosed technology the radial mode acoustic resonator (such as resonator 802 shown in FIG. 8) may be configured with combined piezoelectric layers and metal layers having a thickness ranging from 1 micron to 20 microns. In certain exemplary implementations, the thickness may range from 20 micros to 40 microns.

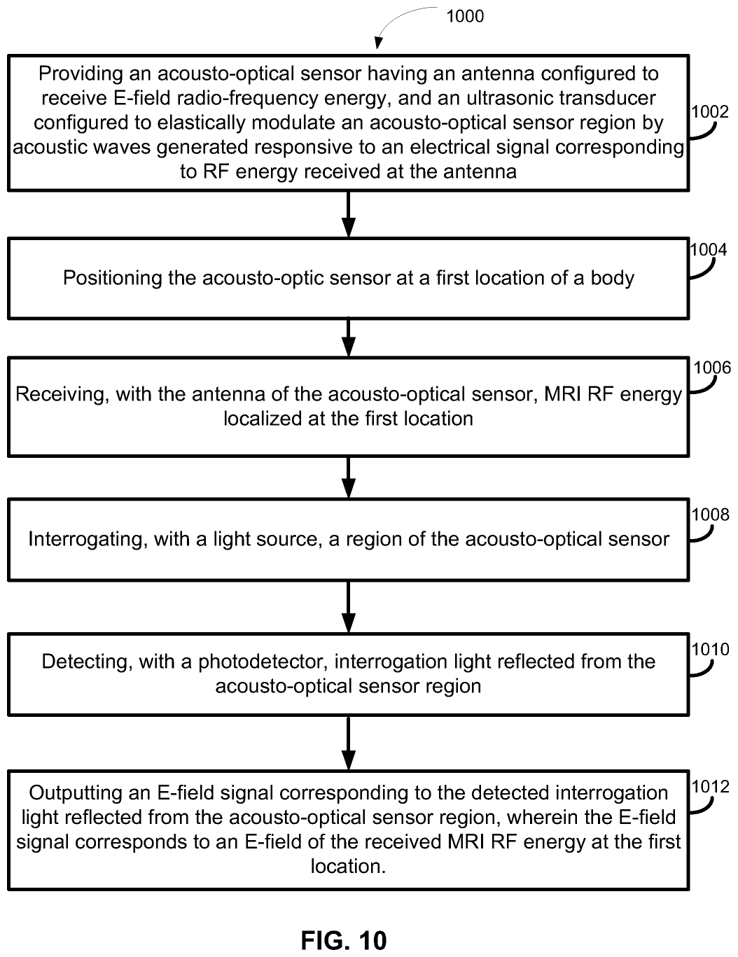

FIG. 10 is a flow diagram of a method 1000 of measuring a local E-field during an MRI scan, according to an exemplary embodiment of the disclosed technology. In block 1002, the method 1000 includes providing an acousto-optical sensor having an antenna configured to receive E-field radio-frequency energy, and an ultrasonic transducer configured to elastically modulate an acousto-optical sensor region by acoustic waves generated responsive to an electrical signal corresponding to RF energy received at the antenna. In block 1004, the method 1000 includes positioning the acousto-optical sensor at a first location of a body. In block 1006, the method 1000 includes receiving, with an antenna of the acousto-optical sensor, MRI RF energy localized at the first location. In block 1008, the method 1000 includes interrogating, with a light source, a region of the acousto-optical sensor. In block 1010, the method 1000 includes detecting, with a photodetector, interrogation light reflected from the acousto-optical sensor region. In block 1012, the method 1000 includes outputting an E-field signal corresponding to the detected interrogation light reflected from the acousto-optical sensor region, wherein the E-field signal corresponds to an E-field of the received MRI RF energy at the first location.

In certain exemplary implementations, the antenna is selected to be one of a dipole antenna, a monopole antenna, or a microstrip antenna.

Certain implementations can include calibrating the acousto-optical sensor, which can include receiving, with the acousto-optical sensor, test RF energy having a known field strength; outputting a test signal corresponding to detected interrogation light reflected from the acousto-optical sensor region, wherein the test signal corresponds to the received test RF energy; determining a calibration coefficient, the calibration coefficient comprising a ratio of the test signal amplitude and the known field strength, and applying the calibration coefficient to the E-field signal to produce a calibrated output signal.

In certain exemplary implementations, the first location is on the surface of the body. In other implementations, the first location is within the body. In certain exemplary implementations, the body is a human patient or a phantom.

According to an exemplary implementation of the disclosed technology, the acousto-optical sensor region comprises a fiber Bragg grating (FBG).

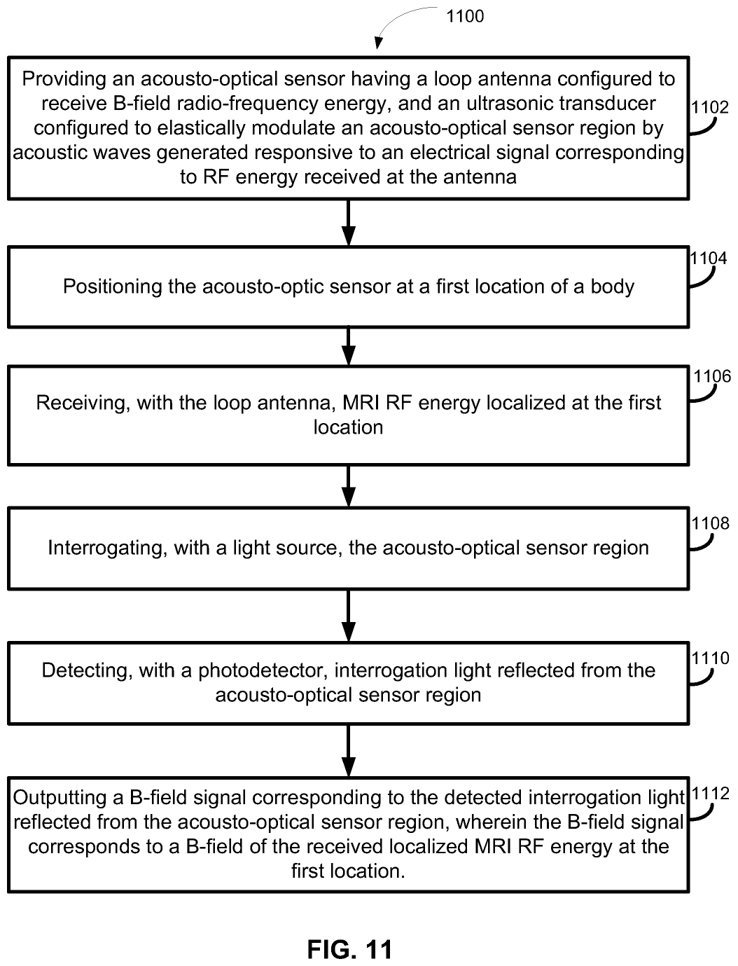

FIG. 11 is a flow diagram of a method 1100 of measuring a local B-field during an MRI scan, according to an exemplary embodiment of the disclosed technology. In block 1102, the method 1100 includes providing an acousto-optical sensor having a loop antenna configured to receive B-field radio-frequency energy, and an ultrasonic transducer configured to elastically modulate an acousto-optical sensor region by acoustic waves generated responsive to an electrical signal corresponding to RF energy received at the loop antenna. In block 1104, the method 1100 includes positioning the acousto-optical sensor at a first location of a body. In block 1106, the method 1100 includes receiving, with the loop antenna, MRI RF energy localized at the first location. In block 1108, the method 1100 includes interrogating, with a light source, the acousto-optical sensor region. In block 1110, the method 1100 includes detecting, with a photodetector, interrogation light reflected from the acousto-optical sensor region. In block 1112, the method 1100 includes outputting a B-field signal corresponding to the detected interrogation light reflected from the acousto-optical sensor region, wherein the B-field signal corresponds to a B-field of the received localized MRI RF energy at the first location.

Certain implementations can include calibrating the acousto-optical sensor, which can include receiving, with the acousto-optical sensor, test RF energy having a known field strength; outputting a test signal corresponding to detected interrogation light reflected from the acousto-optical sensor region, wherein the test signal corresponds to the received test RF energy; determining a calibration coefficient, the calibration coefficient comprising a ratio of the test signal amplitude and the known field strength, and applying the calibration coefficient to the B-field signal to produce a calibrated output signal.

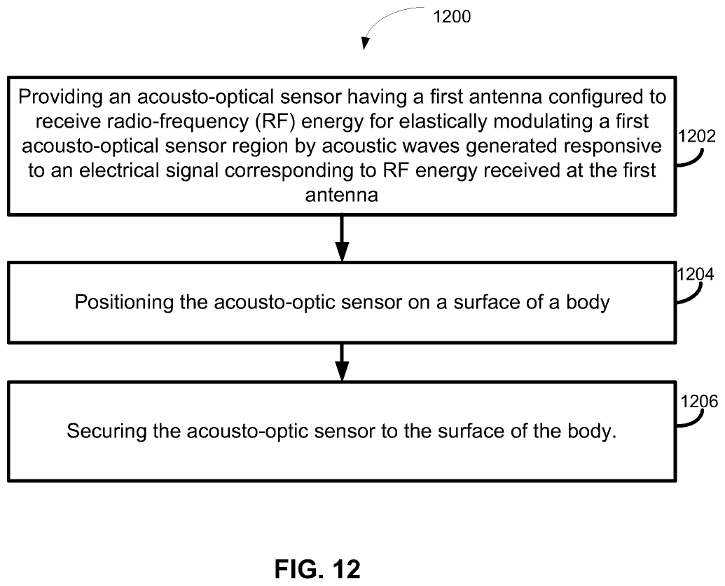

FIG. 12 is a flow diagram of a method 1200 of attaching one or more acousto-optical sensors on a surface of a body, according to an exemplary embodiment of the disclosed technology. In block 1202, the method 1200 includes providing an acousto-optical sensor having a first antenna configured to receive radio-frequency (RF) energy for elastically modulating a first acousto-optical sensor region by acoustic waves generated responsive to an electrical signal corresponding to RF energy received at the first antenna. In block 1204, the method 1200 includes positioning the acousto-optical sensor on a surface of a body. In block 1206, the method 1200 includes securing the acousto-optical sensor to the surface of the body.

In certain exemplary implementations, securing the acousto-optical sensor to the surface of the body can include covering at least a portion of the acousto-optical sensor and at least a portion of the surface of the body with biocompatible adhesive tape.

In certain exemplary implementations, the first antenna is a loop antenna configured to receive localized MRI B-field RF energy. The first antenna is chosen from one of a dipole antenna, a multipole antenna, or a microstrip antenna configured to receive localized MRI E-field RF energy.

Certain exemplary implementations may include providing a second antenna configured to receive radio-frequency (RF) energy for elastically modulating a second acousto-optical sensor region by acoustic waves generated responsive to an electrical signal corresponding to RF energy received at the second antenna. In certain exemplary implementations, the second antenna is a loop antenna configured to receive localized MRI B-field RF energy. In certain exemplary implementations, the first antenna is chosen from one of a dipole antenna, a multipole antenna, or a microstrip antenna configured to receive localized MRI E-field RF energy.

In certain exemplary implementations, providing the acousto-optical sensor can further include providing: a second optical fiber including a distal end; a second acousto-optical sensor region disposed towards the distal end of the second optical fiber, the second acousto-optical sensor region including an electro-mechanical conversion assembly comprising: a second antenna configured to receive radio-frequency (RF) energy and to produce a corresponding second electrical signal; and a second ultrasonic transducer in mechanical communication with the second acousto-optical sensor region, wherein the second ultrasonic transducer is in electrical communication with the second antenna, and wherein the second ultrasonic transducer is configured to elastically modulate the second acousto-optical sensor region by acoustic waves generated responsive to the electrical signal received at the second antenna. In certain exemplary implementations, the second antenna may be a loop antenna configured to receive localized MRI B-field RF energy. In certain exemplary implementations, the first antenna is chosen from one of a dipole antenna, a multipole antenna, or a microstrip antenna configured to receive localized MRI E-field RF energy.

In certain exemplary implementations, the acousto-optical sensor regions include a fiber Bragg grating (FBG).

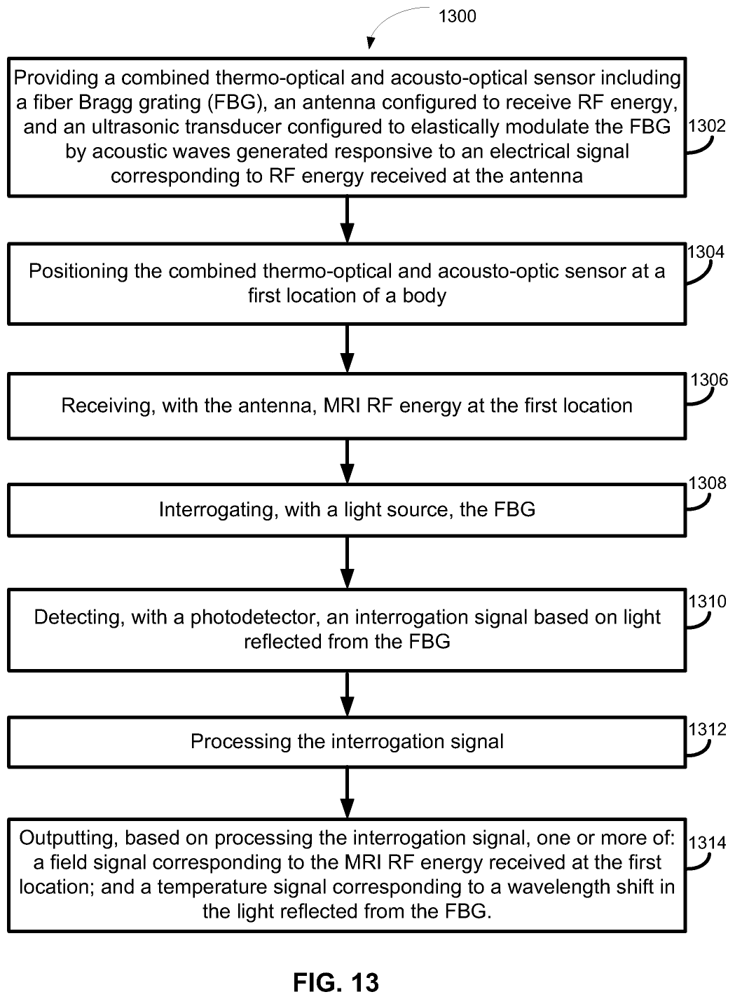

FIG. 13 is a flow diagram of a method 1300 of measuring a local temperature and one or more of a local E-field or local B-field during an MRI scan, according to an exemplary embodiment of the disclosed technology. In block 1302, the method 1300 includes providing a combined thermo-optical and acousto-optical sensor including a fiber Bragg grating (FBG), an antenna configured to receive RF energy, and an ultrasonic transducer configured to elastically modulate the FBG by acoustic waves generated responsive to an electrical signal corresponding to RF energy received at the antenna. In block 1304, the method 1300 includes positioning the combined thermo-optical and acousto-optical sensor at a first location of a body. In block 1306, the method 1300 includes receiving, with the antenna, MRI RF energy at the first location. In block 1308, the method 1300 includes interrogating, with a light source, the FBG. In block 1310, the method 1300 includes detecting, with a photodetector, an interrogation signal based on light reflected from the FBG. In block 1312, the method 1300 includes processing the interrogation signal. In block 1314, the method 1300 includes outputting, based on processing the interrogation signal, one or more of: a field signal corresponding to the MRI RF energy received at the first location; and a temperature signal corresponding to a wavelength shift in the light reflected from the FBG.

In certain exemplary implementations, the antenna is a loop antenna configured to receive localized MRI B-field RF energy, and wherein the field signal corresponds to the localized MRI B-field RF energy.

In certain exemplary implementations, the antenna is chosen from one of a dipole antenna, a multipole antenna, or a microstrip antenna configured to receive localized MRI E-field RF energy, and wherein the field signal corresponds to the localized MRI E-field RF energy.

In certain exemplary implementations, the temperature signal corresponds to a slowly varying wavelength shift in the light reflected from the FBG, wherein the slowly varying wavelength shift is characterized by a component of the interrogation signal having a bandwidth less than 100 Hz, and wherein the slowly varying wavelength shift corresponds to one or more of a thermal expansion and a thermally-induced refractive index change of the FBG.

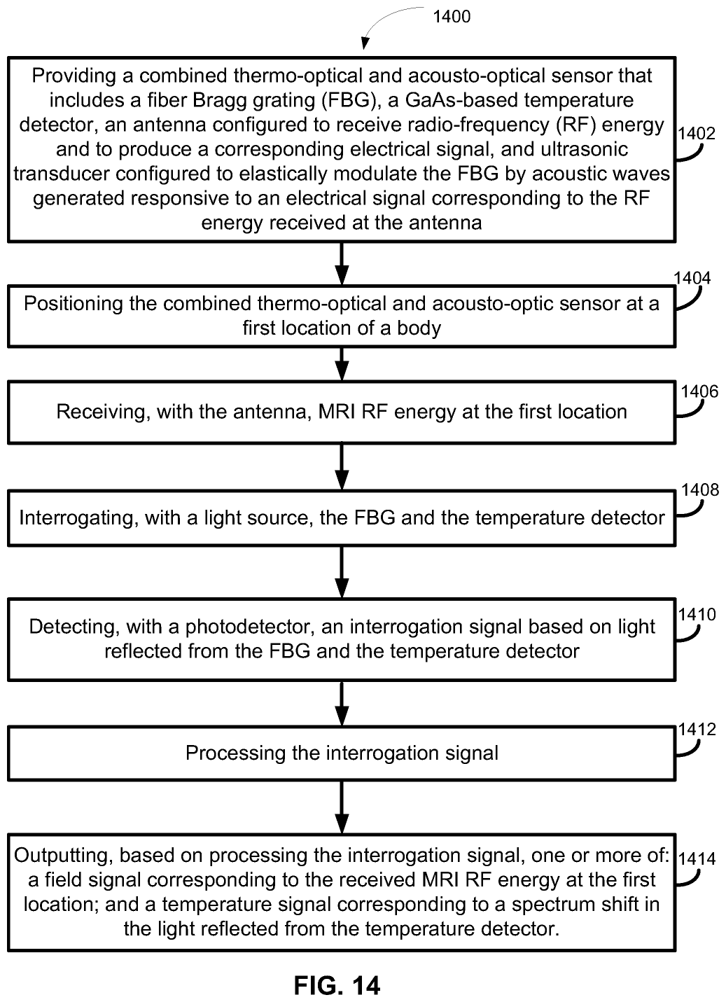

FIG. 14 is a flow diagram of a method 1400 of measuring a local temperature and one or more of a local E-field or local B-field during an MRI scan, according to an exemplary embodiment of the disclosed technology. In block 1402, the method 1400 includes providing a combined thermo-optical and acousto-optical sensor that includes a fiber Bragg grating (FBG), a GaAs-based temperature detector, an antenna configured to receive radio-frequency (RF) energy and to produce a corresponding electrical signal, and an ultrasonic transducer configured to elastically modulate the FBG by acoustic waves generated responsive to an electrical signal corresponding to the RF energy received at the antenna. In block 1404, the method 1400 includes positioning the combined thermo-optical and acousto-optical sensor at a first location of a body. In block 1406, the method 1400 includes receiving, with the antenna, MRI RF energy at the first location. In block 1408, the method 1400 includes interrogating, with a light source, the FBG, and the temperature detector. In block 1410, the method 1400 includes detecting, with a photodetector, an interrogation signal based on light reflected from the FBG and the temperature detector. In block 1412, the method 1400 includes processing the interrogation signal. In block 1414, the method 1400 includes outputting, based on processing the interrogation signal, one or more of: a field signal corresponding to the received MRI RF energy at the first location, and a temperature signal corresponding to a spectrum shift in the light reflected from the temperature detector.

In certain exemplary implementations, the antenna is a loop antenna configured to receive localized MRI B-field RF energy, and wherein the field signal corresponds to the localized MRI B-field RF energy.

In certain exemplary implementations, the antenna is chosen from one of a dipole antenna, a multipole antenna, or a microstrip antenna configured to receive localized MRI E-field RF energy, and wherein the field signal corresponds to the localized MRI E-field RF energy.

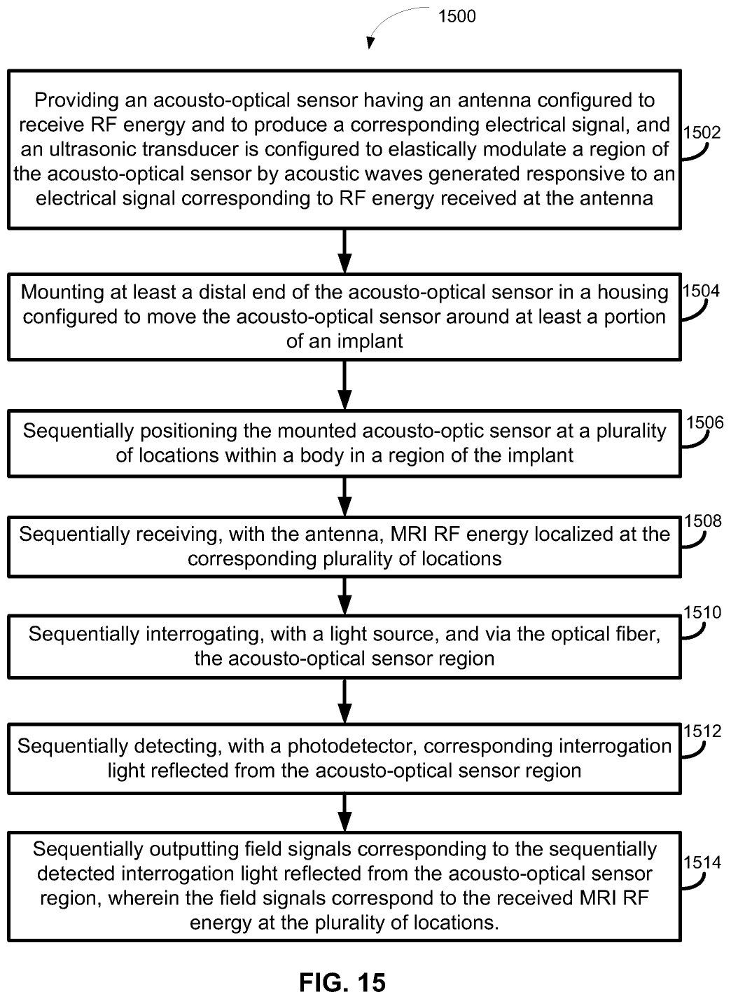

FIG. 15 is a flow diagram of a method 1500 of mapping effects of MRI over an implant, according to an exemplary embodiment of the disclosed technology. In block 1502, the method 1500 includes providing an acousto-optical sensor having an antenna configured to receive RF energy and to produce a corresponding electrical signal, and an ultrasonic transducer is configured to elastically modulate a region of the acousto-optical sensor by acoustic waves generated responsive to an electrical signal corresponding to RF energy received at the antenna. In block 1504, the method 1500 includes mounting at least a distal end of the acousto-optical sensor in a housing configured to move the acousto-optical sensor around at least a portion of an implant. In block 1506, the method 1500 includes sequentially positioning the mounted acousto-optical sensor at a plurality of locations within a body in a region of the implant. In block 1508, the method 1500 includes sequentially receiving, with the antenna, MRI RF energy localized at the corresponding plurality of locations. In block 1510, the method 1500 includes sequentially interrogating, with a light source, and via the optical fiber, the acousto-optical sensor region. In block 1514, the method 1500 includes sequentially detecting, with a photodetector, corresponding interrogation light reflected from the acousto-optical sensor region. In block 1514, the method 1500 includes sequentially outputting field signals corresponding to the sequentially detected interrogation light reflected from the acousto-optical sensor region, wherein the field signals correspond to the received MRI RF energy at the plurality of locations.

In certain exemplary implementations, the antenna is a loop antenna configured to receive localized MRI B-field RF energy, and wherein the field signals correspond to localized MRI B-field RF energy measurements at the corresponding plurality of locations.

In certain exemplary implementations, the antenna is chosen from one of a dipole antenna, a multipole antenna, or a microstrip antenna configured to receive localized MRI E-field RF energy, and wherein the field signals correspond to the localized MRI E-field RF energy measurements at the corresponding plurality of locations.

In certain exemplary implementations, the acousto-optical sensor comprises a fiber Bragg grating (FBG).

Certain exemplary implementations can further include: processing the field signals; and outputting, based on processing the field signals, a plurality of temperature signals corresponding to the plurality of locations, wherein the temperature signals corresponds to a slowly varying wavelength shift in the light reflected from the FBG.

In certain exemplary implementations, the acousto-optical sensor may be disposed in housing is characterized by a tube shape.

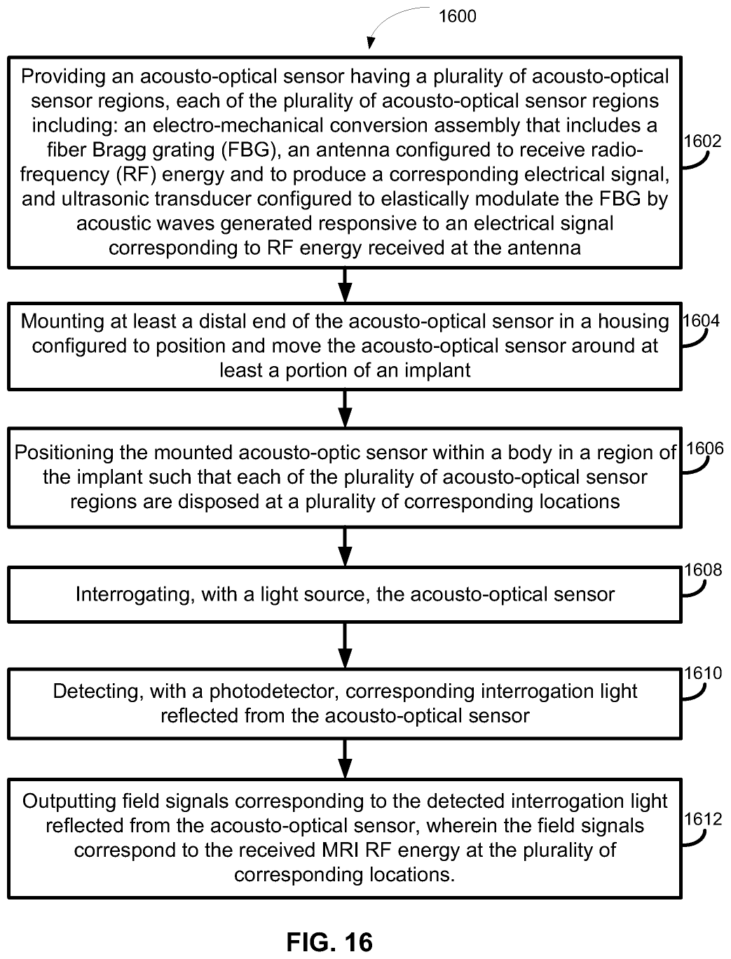

FIG. 16 is a flow diagram of a method 1600 of simultaneously mapping effects of MRI over an implant, according to an exemplary embodiment of the disclosed technology. In block 1602, the method 1600 includes providing an acousto-optical sensor having a plurality of acousto-optical sensor regions, each of the plurality of acousto-optical sensor regions including an electro-mechanical conversion assembly that includes a fiber Bragg grating (FBG), an antenna configured to receive radio-frequency (RF) energy and to produce a corresponding electrical signal, and an ultrasonic transducer configured to elastically modulate the FBG by acoustic waves generated responsive to an electrical signal corresponding to RF energy received at the antenna. In block 1604, the method 1600 includes mounting at least a distal end of the acousto-optical sensor in a housing configured to position and move the acousto-optical sensor around at least a portion of the implant. In block 1606, the method 1600 includes positioning the mounted acousto-optical sensor within a body in a region of the implant such that each of the plurality of acousto-optical sensor regions are disposed at a plurality of corresponding locations. In block 1608, the method 1600 includes interrogating, with a light source, the acousto-optical sensor. In block 1610, the method 1600 includes detecting, with a photodetector, corresponding interrogation light reflected from the acousto-optical sensor. In block 1612, the method 1600 includes outputting field signals corresponding to the detected interrogation light reflected from the acousto-optical sensor, wherein the field signals correspond to the received MRI RF energy at the plurality of corresponding locations.

In certain exemplary implementations, the antenna is a loop antenna configured to receive localized MRI B-field RF energy, and wherein the field signals correspond to localized MRI B-field RF energy measurements at the plurality of corresponding locations.

In certain exemplary implementations, the antenna is chosen from one of a dipole antenna, a multipole antenna, or a microstrip antenna configured to receive localized MRI E-field RF energy, and wherein the field signals correspond to the localized MRI E-field RF energy measurements at the plurality of corresponding locations.

In accordance with certain exemplary implementations of the disclosed technology, the method 1600 can further include processing the field signals; and outputting, based on processing the field signals, a plurality of temperature signals corresponding to the plurality of locations, wherein the temperature signals corresponds to a slowly varying wavelength shift in the light reflected from the FBG.

Certain exemplary implementations of the disclosed technology may include an optical fiber having a distal end; an acousto-optical sensor region disposed at the distal end of the optical fiber; an electro-mechanical conversion assembly in communication with the acousto-optical sensor region, the electro-mechanical conversion assembly including: one or more antennae disposed on the mounting tube structure, the one or more antennae configured to receive radio-frequency (RF) electromagnetic energy and produce a corresponding electrical signal; and an ultrasonic transducer in mechanical communication with the acousto-optical sensor region, wherein the ultrasonic transducer is in electrical communication with the one or more antennae, and wherein the ultrasonic transducer is configured to elastically modulate the acousto-optical sensor region by acoustic waves generated responsive to the electrical signal received from the one or more antennae.

In certain implementations, the device can include a resonator feature in communication with the optical fiber, wherein the resonator feature is configured to at least partially reflect the generated acoustic waves for enhanced modulation of the acousto-optical sensor region. In certain exemplary implementations, the resonator feature can include an acoustical discontinuity comprising a notch formed by removal of at least a portion of cladding from the optical fiber. In certain exemplary implementations, the resonator feature can include an acoustical discontinuity comprising deposition of a ring of material on the optical fiber. Yet, in certain implementations, the resonator uses radial vibration resonances of the optical fiber under the piezoelectric transducer over the FBG region.

In accordance with certain implementations of the disclosed technology, the one or more antennae can include at least a first antenna and a second antenna, wherein the second antenna may be oriented in an orthogonal direction with respect to the first antenna.

In certain exemplary implementations, the acousto-optical sensor region can include a first fiber Bragg grating (FBG) and a second FBG. In certain exemplary implementations, the electromagnetic conversion assembly can include a first piezoelectric transducer in mechanical communication with the first FBG and in electrical communication with the first antenna. The electromagnetic conversion assembly can include a second piezoelectric transducer in mechanical communication with the second FBG and in electrical communication with the second antenna.

In accordance with certain implementations of the disclosed technology, the one or more antennae can include one or more of: a patch antenna; a coil antenna; a loop antenna, a monopole antenna, a microstrip antenna, and a dipole antenna.

In accordance with certain exemplary implementations of the disclosed technology, the acousto-optical sensor region can include a fiber Bragg grating (FBG).

In accordance with certain exemplary implementations of the disclosed technology, the ultrasonic transducer can include a piezoelectric transducer.

In accordance with certain exemplary implementations of the disclosed technology, the optical fiber can include at least one proximal end configured for coupling with an external light source for interrogation of the acousto-optical sensor region.

In accordance with certain exemplary implementations of the disclosed technology, the optical fiber can include at least one proximal end configured for coupling with a photodetector to receive interrogation light reflected from the acousto-optical sensor region.

In accordance with certain exemplary implementations of the disclosed technology, the optical fiber and the electro-mechanical conversion assembly are configured to reduce MRI RF-induced heating of the device.

In certain exemplary implementations, acousto-optical sensor region and/or the optical fiber may include a resonator feature that can be configured to at least partially reflect the generated acoustic waves to enhance a modulation amplitude of the acousto-optical sensor region. In certain implementations, the resonator feature includes an acoustical discontinuity that can include one or more of: a notch formed by removal of at least a portion of cladding from the optical fiber, and deposition of a material on the optical fiber. In some embodiments, the piezoelectric thin film transducer on the fiber can serve as the resonator using the radial vibration modes of the composite optical fiber/thin film transducer structure.

In certain implementations, the acousto-optical sensor region can include a first fiber Bragg grating (FBG) and a second FBG. In certain exemplary embodiments, the electromagnetic conversion assembly can include a first piezoelectric transducer in mechanical communication with the first FBG and in electrical communication with one or more of the first antenna and the second antenna. In certain example implementations, the electromagnetic conversion assembly can include a second piezoelectric transducer in mechanical communication with the second FBG and in electrical communication with the second antenna.

Numerous specific details of the disclosed technology are set forth herein. However, it is to be understood that implementations of the disclosed technology may be practiced without these specific details. In other instances, well-known methods, structures, and techniques have not been shown in detail in order not to obscure an understanding of this description. References to “one implementation,” “an implementation,” “exemplary implementation,” “various implementations,” etc., indicate that the implementation(s) of the disclosed technology so described may include a particular feature, structure, or characteristic, but not every implementation necessarily includes the particular feature, structure, or characteristic. Further, repeated use of the phrase “in one implementation” does not necessarily refer to the same implementation, although it may. The use of “exemplary” herein carries the same meaning as “example,” and is not intended to mean “preferred” or “best.”

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form.

As used herein, unless otherwise specified the use of the ordinal adjectives “first,” “second,” “third,” etc., to describe a common object, merely indicate that different instances of like objects are being referred to and are not intended to imply that the objects so described must be in a given sequence, either temporally, spatially, in ranking, or in any other manner.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described as making up the various elements of the invention are intended to be illustrative and not restrictive. Many suitable materials that would perform the same or a similar function as the materials described herein are intended to be embraced within the scope of the invention. Such other materials not described herein can include but are not limited to, for example, materials that are developed after the time of the development of the invention.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and to enable any person skilled in the art to practice certain implementations of the disclosed technology, including making and using any devices or systems and performing any incorporated methods. The patentable scope of certain implementations of the disclosed technology is defined in the claims and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method of measuring a local field during an MRI scan, the method comprising:
positioning an acousto-optical sensor at a first location of a body, wherein the acousto-optical sensor comprises:
an optical fiber including a distal end; and
an acousto-optical sensor region disposed towards the distal end of the optical fiber, the acousto-optical sensor region including an electro-mechanical conversion assembly comprising:
an antenna configured to receive field radio-frequency (RF) energy and to produce a corresponding electrical signal; and
an ultrasonic transducer in mechanical communication with the acousto-optical sensor region, wherein the ultrasonic transducer is in electrical communication with the antenna, and wherein the ultrasonic transducer is configured to elastically modulate the acousto-optical sensor region by acoustic waves generated responsive to an electrical signal received from the antenna that corresponds to RF energy received by the antenna;
receiving, with the antenna, MRI RF energy localized at the first location;
interrogating, with a light source, and via the optical fiber, the acousto-optical sensor region;
detecting, with a photodetector, interrogation light reflected from the acousto-optical sensor region; and
outputting a signal corresponding to the detected interrogation light reflected from the acousto-optical sensor region;
wherein the signal corresponds to a field of the received MRI RF energy at the first location.

2. The method of claim 1, wherein the local field is a local E-field;
wherein the output signal is an E-field signal;
wherein the E-field signal corresponds to an E-field of the received MRI RF energy;
wherein the antenna is configured to receive E-field RF energy; and
wherein the antenna is selected from the group consisting of a dipole antenna, a monopole antenna, and a microstrip antenna.

3. The method of claim 2 further comprising calibrating the acousto-optical sensor comprising:
receiving, with the acousto-optical sensor, test RF energy having a known field strength;
outputting a test signal corresponding to detected interrogation light reflected from the acousto-optical sensor region, wherein the test signal corresponds to the received test RF energy;
determining a calibration coefficient, the calibration coefficient comprising a ratio of an amplitude of the test signal and the known field strength; and
applying the calibration coefficient to the E-field signal to produce a calibrated output signal.

4. The method of claim 2, wherein the first location is selected from the group consisting of a location on a surface of the body and a location within the body.

5. (canceled)

6. The method of claim 2, wherein the body is a human patient or a phantom.

7. The method of claim 2, wherein the acousto-optical sensor region comprises a fiber Bragg grating (FBG).

8. The method of of claim 1, wherein the local field is a local B-field;
wherein the output signal field is a B-field signal;
wherein the B-field signal corresponds to a B-field of the received MRI RF energy;
wherein the antenna is configured to receive B-field RF energy; and
wherein the antenna is a loop antenna.

9. The method of claim 8 further comprising calibrating the acousto-optical sensor, the calibrating comprising:
receiving, with the acousto-optical sensor, test RF energy having a known field strength;
outputting a test signal corresponding to detected interrogation light reflected from the acousto-optical sensor region, wherein the test signal corresponds to the received test RF energy;
determining a calibration coefficient, the calibration coefficient comprising a ratio of an amplitude of the test signal amplitude and the known field strength; and
outputting the calibration coefficient.

10. The method of claim 9 further comprising applying the calibration coefficient to the B-field signal to produce a calibrated output signal.

11. The method of claim 8, wherein the first location is selected from the group consisting of a location on a surface of the body and a location within the body; and
wherein the body is a human patient or a phantom.

12. The method of claim 8, wherein the acousto-optical sensor region comprises a fiber Bragg grating (FBG).

13. A method of attaching one or more acousto-optical sensors on a surface of a body, the method comprising:
positioning an acousto-optical sensor on a surface of a body; and
securing the acousto-optical sensor to the surface of the body;
wherein the acousto-optical sensor comprises:
a first optical fiber including a distal end; and
a first acousto-optical sensor region disposed at a first position towards the distal end of the first optical fiber, the first acousto-optical sensor region including an electro-mechanical conversion assembly comprising:
a first antenna configured to receive radio-frequency (RF) energy and to produce a corresponding first electrical signal; and
a first ultrasonic transducer in mechanical communication with the first acousto-optical sensor region, wherein the first ultrasonic transducer is in electrical communication with the first antenna, and wherein the first ultrasonic transducer is configured to elastically modulate the first acousto-optical sensor region by acoustic waves generated responsive to an electrical signal received from the first antenna that corresponds to RF energy received by the first antenna.

14. The method of claim 13, wherein securing the acousto-optical sensor to the surface of the body comprises covering at least a portion of the acousto-optical sensor and at least a portion of the surface of the body with biocompatible adhesive tape.

15. The method of claim 13, wherein the first antenna is selected from the group consisting of a loop antenna, a dipole antenna, a multipole antenna, and a microstrip antenna;
wherein the loop antenna is configured to receive localized MRI B-field RF energy; and
wherein the dipole antenna, the multipole antenna, and the microstrip antenna configured to receive localized MRI E-field RF energy.

16. (canceled)

17. The method of claim 13, wherein the acousto-optical sensor further comprises:
a second acousto-optical sensor region disposed at a second position towards the distal end of the first optical fiber, the second acousto-optical sensor region including an electro-mechanical conversion assembly comprising:
a second antenna configured to receive radio-frequency (RF) energy and to produce a corresponding second electrical signal; and
a second ultrasonic transducer in mechanical communication with the second acousto-optical sensor region, wherein the second ultrasonic transducer is in electrical communication with the second antenna, and wherein the second ultrasonic transducer is configured to elastically modulate the second acousto-optical sensor region by acoustic waves generated responsive to an electrical signal received from the second antenna that corresponds to RF energy received by the second antenna.

18. The method of claim 17, wherein the second antenna is a loop antenna configured to receive localized MRI B-field RF energy; and
wherein the first antenna is selected from the group consisting of a dipole antenna, a multipole antenna, and a microstrip antenna, each configured to receive localized MRI E-field RF energy.

19. The method of claim 13, wherein the acousto-optical sensor further comprises:
a second optical fiber including a distal end; and
a second acousto-optical sensor region disposed towards the distal end of the second optical fiber, the second acousto-optical sensor region including an electro-mechanical conversion assembly comprising:
a second antenna configured to receive radio-frequency (RF) energy and to produce a corresponding second electrical signal; and
a second ultrasonic transducer in mechanical communication with the second acousto-optical sensor region, wherein the second ultrasonic transducer is in electrical communication with the second antenna, and wherein the second ultrasonic transducer is configured to elastically modulate the second acousto-optical sensor region by acoustic waves generated responsive to an electrical signal received from the second antenna that corresponds to RF energy received by the second antenna.

20. The method of claim 19, wherein the second antenna is a loop antenna configured to receive localized MRI B-field RF energy; and
wherein the first antenna is selected from the group consisting of a dipole antenna, a multipole antenna, and a microstrip antenna, each configured to receive localized MRI E-field RF energy.

21. The method of claim 13, wherein the acousto-optical sensor region comprises a fiber Bragg grating (FBG).

22. A method of measuring a local temperature and one or more of a local E-field or local B-field during an MRI scan, the method comprising:
positioning a combined thermo-optical and acousto-optical sensor at a first location of a body, wherein the combined thermo-optical and acousto-optical sensor comprises:
an optical fiber including a distal end;
a fiber Bragg grating (FBG) disposed towards the distal end of the optical fiber; and
an electro-mechanical conversion assembly comprising:
an antenna configured to receive radio-frequency (RF) energy and to produce a corresponding electrical signal; and
an ultrasonic transducer in mechanical communication with the FBG, wherein the ultrasonic transducer is in electrical communication with the antenna, and wherein the ultrasonic transducer is configured to elastically modulate the FBG by acoustic waves generated responsive to an electrical signal received from the antenna that corresponds to RF energy received by the antenna;
receiving, with the antenna, MRI RF energy at the first location;
interrogating, with a light source, and via the optical fiber, the FBG;
detecting, with a photodetector, an interrogation signal based on light reflected from the FBG;
processing the interrogation signal; and
outputting, based on processing the interrogation signal, one or more of:
a field signal corresponding to the received MRI RF energy at the first location; and
a temperature signal corresponding to a wavelength shift in the light reflected from the FBG.

23.-24. (canceled)

25. The method of claim 22, wherein the temperature signal corresponds to a slowly varying wavelength shift in the light reflected from the FBG;
wherein the slowly varying wavelength shift is characterized by a component of the interrogation signal having a bandwidth less than 100 Hz; and
wherein the slowly varying wavelength shift corresponds to one or more of a thermal expansion and a thermally-induced refractive index change of the FBG.

26. The method of claim 22, wherein the combined thermo-optical and acousto-optical sensor further comprises a GaAs-based temperature detector disposed at the distal end of the optical fiber;
wherein interrogating, with the light source, and via the optical fiber, further comprises interrogating the temperature detector;
wherein the interrogation signal is further based on light reflected from the temperature detector; and
wherein the outputting further comprises the option of a temperature signal corresponding to a spectrum shift in the light reflected from the temperature detector.

27.-28. (canceled)

29. A method of mapping effects of MRI over an implant, the method comprising:
mounting at least a distal end of an acousto-optical sensor in a housing configured to move the acousto-optical sensor around at least a portion of the implant, wherein the acousto-optical sensor comprises:
an optical fiber including a distal end; and
an acousto-optical sensor region disposed towards the distal end of the optical fiber, the acousto-optical sensor region including an electro-mechanical conversion assembly comprising:
an antenna configured to receive radio-frequency (RF) energy and to produce a corresponding electrical signal; and
an ultrasonic transducer in mechanical communication with the acousto-optical sensor region, wherein the ultrasonic transducer is in electrical communication with the antenna, and wherein the ultrasonic transducer is configured to elastically modulate the acousto-optical sensor region by acoustic waves generated responsive to an electrical signal received from the antenna that corresponds to RF energy received by the antenna;
sequentially positioning the mounted acousto-optical sensor at a plurality of locations within a body in a region of the implant;
sequentially receiving, with the antenna, MRI RF energy localized at the corresponding plurality of locations;
sequentially interrogating, with a light source, and via the optical fiber, the acousto-optical sensor region;
sequentially detecting, with a photodetector, corresponding interrogation light reflected from the acousto-optical sensor region; and
sequentially outputting field signals corresponding to the sequentially detected interrogation light reflected from the acousto-optical sensor region, wherein the field signals correspond to the received MRI RF energy at the plurality of locations.

30.-32. (canceled)

33. The method of claim 29, wherein the acousto-optical sensor comprises a fiber Bragg grating (FBG); and
wherein the method further comprises:
processing the field signals; and
outputting, based on processing the field signals, a plurality of temperature signals corresponding to the plurality of locations, wherein the temperature signals corresponds to a slowly varying wavelength shift in the light reflected from the FBG.

34.-35. (canceled)

36. A method of simultaneously mapping effects of MRI over an implant, the method comprising:
mounting at least a distal end of an acousto-optical sensor in a housing configured to position and move the acousto-optical sensor around at least a portion of the implant, wherein the acousto-optical sensor comprises:
an optical fiber including a distal end; and
a plurality of acousto-optical sensor regions disposed towards the distal end of the optical fiber, each of the plurality of acousto-optical sensor regions including an electro-mechanical conversion assembly comprising:
a fiber Bragg grating (FBG);
an antenna configured to receive radio-frequency (RF) energy and to produce a corresponding electrical signal; and
an ultrasonic transducer in mechanical communication with the FBG wherein the ultrasonic transducer is in electrical communication with the antenna, and wherein the ultrasonic transducer is configured to elastically modulate the FBG by acoustic waves generated responsive to an electrical signal received from the antenna that corresponds to RF energy received by the antenna;
positioning the mounted acousto-optical sensor within a body in a region of the implant such that each of the plurality of acousto-optical sensor regions are disposed at a plurality of corresponding locations;
receiving, with each antenna of the plurality of acousto-optical sensor regions, MRI RF energy localized at the plurality of corresponding locations;
interrogating, with a light source, and via the optical fiber, the acousto-optical sensor;
detecting, with a photodetector, corresponding interrogation light reflected from the acousto-optical sensor; and
outputting field signals corresponding to the detected interrogation light reflected from the acousto-optical sensor, wherein the field signals correspond to the received MRI RF energy at the plurality of corresponding locations.

37.-40. (canceled)
